(19) United States
(12) Patent Application Publication
(10) Pub. No.: US 2003/0065019 A1
(43) Pub. Date: Apr. 3, 2003

(54) Treatment of rheumatoid arthritis with statins (HMG-CoA reductase inhibitors)

(76) Inventor: Francois Mach, Vesenaz (CH)

Correspondence Address: MINTZ, LEVIN, COHN, FERRIS,, GLOVSKY and POPEO, P.C., One Financial Center, Boston, MA, 02111, US

(21) Appl. No.: 10/056,646

(22) Filed: Jan. 23, 2002

Related U.S. Application Data
(62) Division of application No. 09/960,471, filed on Sep. 19, 2001.

(51) Int. Cl. A61K 31/401; A61K 31/366; A61K 31/225

(52) U.S. Cl. 514/423; 514/548; 514/460

(57) ABSTRACT

The present invention relates to methods of causing MHC-class II or CD40 mediated immunomodulation, immunosuppression and anti-inflammatory action, in a subject suffering from or susceptible of suffering from a condition involving inappropriate immune response, which comprises administering to the subject at least one statin, or a functionally or structurally equivalent molecule, in an amount effective to modulate MHC class II or CD40 expression in the subject.

### FIELD OF THE INVENTION

The invention relates to the fields of immunology, disease treatment, and more specifically, to the use of immunomodulators to treat autoimmune diseases.

### BACKGROUND OF THE INVENTION

Statins are a new family of molecules sharing the capacity to competitively inhibit the hepatic enzyme 3-hydroxy-3-methylglutaryl coenzyme A (HMG-CoA) reductase. This enzyme catalyses the rate-limiting step in the L-mevalonate pathway for cholesterol synthesis. Consequently, statins block cholesterol synthesis. They are extensively used in medical practice¹⁻³, especially in the treatment of hyperlipidaemia. This class of agent is proving to be effective for preventing heart attacks in patients with hypercholesterolacmia. Moreover, reports of several large clinical trials published during recent years have clearly shown treatment with statins to reduce cardiovascular-related morbidity and mortality in patients with and without coronary disease ^{1-3,8}.

The immune system is highly complex and tightly regulated, with many alternative pathways capable of compensating deficiencies in other parts of the system. There are however occasions when the immune response becomes a cause of disease or other undesirable conditions if activated. Such diseases or undesirable conditions are for example autoimmune diseases (including type I diabetes, multiple sclerosis and rheumatoid arthritis), graft rejection after transplantation, or allergy to innocuous antigens, psoriasis, chronic inflammatory diseases such as atheroslerosis, and inflammation in general. In these cases and others involving inappropriate or undesired immune response there is a clinical need for immunosuppression. The pathways leading to these undesired immune responses are numerous and in many cases are not fully elucidated. However, they often involve a common step which is activation of lymphocytes.

Major Histocompatibility Complex molecules, encoded by the HLA gene cluster in man, are involved in many aspects of immunological recognition, including interaction between different lymphoid cells, as well as between lymphocytes and antigen-presenting cells. Major Histocompatibility Complex class II (MTC class II or MHC-II) molecules are directly involved in the activation of T lymphocytes and in the control of the immune response. Although all cells express class I MHC molecules, class II expression is confined to antigen-presenting cells (APCs). These cells are potentially capable of presenting antigen to lymphocytes T-helper which control the development of an immune response. Thus the expression of MHC class II molecules is the key to antigen presentation. Only a limited number of specialized cell types express MHC class II constitutively, numerous other cells become MHC class II positive upon stimulation. The stimulation is usually induction by a cytokine, particularly by interferon gamma (IFN-γ)⁵.

Regulation of expression of MHC class II genes is highly complex and this tight control directly affects T lymphocyte activation and thus the control of the immune response. This complex regulation bas now been dissected in great detail, thanks to a great extent to a rare human disease of MHC class II regulation, called the Bare Lymphocyte Syndrome (or MHC class II deficiency)⁵. Four groups of patients, all with an identical clinical picture of severe primary immunodeficiency, were shown to be affected genetically in one of four distinct transacting regulatory factors, essential for MHC class II gene transcription: whereas RFX5, RFX-AP or RFX-ANK are ubiquitously expressed factors, forming a protein complex that binds to the X box of MHC class II promoters^{5,10}, CIITA Class II TransActivator) is the general controller of MHC class II expression and its own expression is tightly regulated^{6,7}. Interestingly, expression of CIITA is controlled by several alternative promoters, operating under distinct physiological conditions¹¹. CIITA promoter I controls constitutive expression in dendritic cells, promoter III controls constitutive expression in B and T lymphocytes, while CITTA promoter IV is specifically responsible for the IFN-γ inducible expression of CIITA and thus of MHC class II¹¹. The molecular basis of inducibility of CIITA promoter IV has been elucidated in detail¹².

MHC-II expression is also a key target for alloreactivity of T-lymphocytes in the process of organ rejection following transplantation.

Another molecules triggering activation of lymphocytes are CD40 and CD40L. CD40L (gp39, recently renamed CD154) and CD40 are members of the tumor necrosis factor (EF) and TNF-receptor family, respectively. The original function of CD40L in T cell-dependent humoral immunity involves the activation and differentiation of B-lymphocytes, the switching of immunoglobulin classes, and the formation of germinal center end memory cells. More recently, activation of atheroma associated cells (macrophages [MΦ] endothelial cells [ECs], smooth muscle cells [SMCs]) via CD40 signaling have been shown to induce inflammatory responses with adhesion molecules expression (e.g., E-Selectin, VCAM-1)[Karmann, 1995, 44] [Hollenbaugh, 1995, 45] [Yellin, 1995, 46], secretion of pro-inflammatory cytokines (e.g., IL-1, IL-6, IL-8, IL-12, TNF)[Mach, 1997, 15], matrix metalloproteinases (MMPs) (MMP-1, MMP-9 MMP-13)[Mach, 1997, 47] [Mach, 1999, 48] [Schonbeck, 1997, 49], tissue factor[Mach, 1997, 47] [Schonbock, 2000, 50] and chemokines[Mach, 1999, 51] [Sugiura, 2000, 52].

Atherosclerosis is now considered as an immuno-inflamnatory disease[Libby, 2000, 24] [Lusis, 2000, 26] [Glass, 2001, 27]. According to this view, increasing new evidence suggests a central role for the CD40/CD40L signaling pathway in the process of this disease[Mach, 1998, 28], [Schonbeck, 2001, 29]. Indeed, recent findings have shown that blocking CD40/CD40L interactions significantly prevent the development of atherosclerotic plaques as well as reduce already pre-established lesions [Mach, 1998, 30] [Lutgens, 1999, 37] [Schonbeck, 2000, 38]. CD40 signaling has been implicated in several chronic disorders such as rheumatoid arthritis, multiple sclerosis and allograft rejection after organ transplantation[Durie, 1993, 39] [Gerritse, 1996, 40] [Jensen, 2001, 41] [Shimizu, 2000, 42] [Larsen, 1996, 43].

Rheumatoid arthritis (RA) is the most common inflammatory rheumatic disease affecting approximately 1% of the population. RA is Bated with severe disability and an increased mortality. Histologically, the disease is characterized by synovial hyperplasia and inflammatory cell recruitment, and, in its later stages, cartilage a bone destruction. The presence of a large number of activated T cells in the synovial membrane is a strong evidence that RA is an immune-mediated disease. The role of cytokines such as IL-1 and TNF-α in articular inflammation and in subsequent joint damage has been demonstrated in animal models²¹. The use of cytokine inhibitors in patients with RA led to an improvement of clinical parameters of disease activity and of radiological signs of articular erosions (22,23). Although these novel approaches should be considered as a breakthrough in the management of RA, 30% of patients are resistant to anti-cytokine therapies. It is therefore necessary to find new targets for the treatment of RA.

### SUMMARY OF THE INVENTION

The present invention provides a new class of agents that reduce or repress T-lymphocyte activation mediated by class II or CD40 expression and consequently are capable of acting as inmunomodulators and anti-inflammatory agents.

The mode of action of the agents on the immune system as discovered by the present inventors will be described below, followed by a discussion of the different immune-related applications of statins and the therapeutic uses of these drugs.

In this context, the inventors have demonstrated the following properties of statins in the inhibition of induction of MHC class II expression by IFN-γ and in repression of MHC class II-mediated T cell activation:

First, statins effectively repress the induction of MHC-II expression by IFN-γ and do so in a dose-dependant manner.

Second, in the presence of L-mevalonate (which is the product of the enzyme HMG-CoA reductase, the substrate thereof being HMG-CoA), the effect of statins, on MHC class II expression is abolished, indicating that it is indeed the effect of statins as HMG-CoA reductase inhibitors that mediates repression of MHC class II.

Third, repression of MHC class II expression by statins, is highly specific for the inducible form of MHC-II expression and does not concern constitutive expression of MHC-II in highly specialized APCs, such as dendritic cells and B cells.

Fourth, this effect of statins is specific for MHC class II and does not concern MHC class I expression.

Fifth, pretreatment of endothelial cells with statins represses induction of MHC class II and reduces subsequent T lymphocyte activation and proliferation.

Sixth, the inhibition achieved by statins on CIITA expression is a specific inhibition of the inducible promoter IV of CIITA.

Seventh, statins decrease IFN-γ induced CD40 expression on vascular cells and do so in a dose-dependant manner. This effect is markedly reversed by addition of L-mevalonate.

The novel effect of statins as MHC class II repressor has been observed and confirmed in a number of cell types, including primary cultures of human endothelial cells (ECs), primary human smooth muscle cells, fibroblasts and monocyte-macrophages (Mφ), as well as in established cell lines such as ThP1, melanomas and Hela cells. This effect of statins on MHC class II induction is observed with different forms of statins currently used in clinical medicine. Interestingly however, different statins exhibit quite different potency as MHC class II <<repressors>>. Of Atorvastatin, Lovastatin and Pravastatin, the most powerful MHC class II repressor is Atorvastatin. Other members of the statin family, as well as functionally or structurally related molecules, should lead to the same newly described effect on MHC class II repression.

These results on the mechanism of statin inhibition of MHC class II induction allow to conclude in favor of a selective effect of statins on the induction of expression of promoter IV of the MHC class II transactivator CIITA. Failure to allow inducible expression of MHC class II molecules on the large variety of cells that normally become NMC class II positive under the effect of IFN-γ is expected to have multiple functional consequences. These concern activation of endogenous CD4 T lymphocytes, but also recognition of MHC class II molecules by CD4 T cells in an allogenic context following organ transplantation.

Another aspect of the present invention is directed to a method of treating a patient afflicted with a disease characterized by interferon-gamma mediated stimulation of major histocompatibility class II gene expression, comprising administering to said patient a compound that inhibits 3-hydroxy-3-methylglutaryl coenzyme A (HMG-CoA) reductase in an amount effective to treat said disease.

Another aspect of the present invention is directed to a method of treating a patient afflicted with a disease characterized by interferon-gamma mediated stimulation of major histocompatibility (MHC) class II gene expression, comprising administering to said patient a compound that inhibits 3-hydroxy-3-methylglutaryl coenzyme A (HMG-CoA) reductase in an amount effective reduce MHC class II gene expression.

Another aspect of the present invention is directed to a method of treating a patient afflicted with a disease characterized by interferon-gamma mediated stimulation of Class II transactivator (CIITA) gene expression, comprising administering to said patient a compound that inhibits 3-hydroxy-3-methylglutaryl coenzyme A (HMG-CoA) reductase in an amount effective to treat said disease.

Another aspect of the present invention is directed to a method of treating a patient afflicted with a disease characterized by interferon-gamma mediated stimulation of Class II transactivator (CIITA) gene expression, comprising administering to said patient a compound that inhibits 3-hydroxy-3-methylglutaryl coenzyme A (HMG-CoA) reductase in an amount effective reduce CIITA gene expression.

Another aspect of the present invention is directed to a method of treating a patient suffering from an autoimmune disease or condition comprising:

administering to said patient at least one compound, capable of measurable HMG-CoA reductase inhibition and inhibition of IFN-γ-induced CIITA expression in an IFN-γ responsive cell, in an amount which is effective to treat such autoimmune disease or condition.

Another aspect of the present invention is directed to a method of treating a patient in preparation for or after an organ or tissue transplant comprising:

administering to said patient at least one compound capable of measurable HMG-CoA reductase inhibition and inhibition of IFN-γ-induced CIITA expression in an IFN-γ responsive cell, in an amount which is effective to prevent tissue rejection. In one embodiment, the compound is administered prophylactically to prevent or inhibit the onset of rejection.

### BRIEF DESCRIPTION OF THE DRAWINGS

The invention may be further illustrated by reference to the accompanying drawings wherein:

FIG. 1 is a series of graph panels showing that statins decreased IFN-γ induced MHC class II protein expression on human endothelial cells and macrophages.

FIGS. 1a to 1f are graphs showing flow cytometric analyses for MHC class II proteins (a-e) and MHC class I (f).

FIG. 1a shows flow cytometric analysis achieved on human vascular endothelial cells (ECs) treated with IFN-γ (500 U/ml, 48 hrs) alone (bold line), or with Atorvastatin 10 μM (left dotted line), Lovastatin 10 μM (bold dotted line), or Pravastatin 20 μM (right dotted line).

FIG. 1b shows flow cytometric analysis achieved on ECs treated with IFN-γ (500 U/ml, 48 hrs) alone (bold line), or with Atorvastatin 40 nM, 0.2 μM, 2 μM, or 10 μM (from right to left dotted lines, respectively).

FIG. 1c shows flow cytometric analysis achieved on ECs treated with IFN-γ alone (500 U/ml, 48 hrs) (bold line), or with Atorvastatin (10 μM) and L-mevalonate (100 μM) (dotted line).

FIG. 1d shows flow cytometric analysis achieved on human dendritic cells (DC) under control conditions or treated with Atorvastatin 10 μM (dotted line).

FIG. 1e shows flow cytometric analysis achieved on the human cell line Ragi under control conditions or treated with Atorvastatin (10 μM, 48 hrs)(dotted line).

FIG. 1f shows flow cytometric analysis achieved on ECs treated with IFN-γ (500 U/ml, 48 hrs) alone (bold line), or with Atorvastatin 10 μM (dotted line). For all panels, solid histograms represent MHC class II (a-e) or MHC class I (f) expression under unstimulated conditions. Each panel is a histogram representing cell numbers (y axis) vs. log fluorescence intensity (x axis) for 30,000 viable cells. Similar results were obtained in independent experiments with ECs and DCs from five different donors.

FIG. 1g is a graph showing fluorescence analysis (expressed as relative intensity) for MHC II expression on human macrophages. (1) are cells under unstimulated conditions, (2), (3), (4) and (5) are cells treated with IFN-γ alone (500 U/ml, 48 hrs), or with Atorvastatin (10 μM), Lovastatin (10 M) or Pravastatin (20 μM), respectively. (6) are cells treated with IFN-γ (500 U/ml, 48 hrs) and stained with secondary antibody only (negative control). Similar results were obtained in separate experiments using macrophages from three different donors.

FIG. 2 is the association of a blot and its graphic representation showing that the effect of statins on IFN-γ induced MHC class II expression is mediated by the transactivator CIITA.

FIG. 2a is a reproduction of an RNAse protection assay (RPA) for MHC class II (DR-α) and FIG. 2b is a reproduction of an RNAse protection assay (RPA) for CIITA. Human vascular endothelial cells unstimulated (1), treated with IFN-γ (500 U/ml, 12 hrs) alone (2), or with Atorvastatin (10 μM) (3), Lovastin (10 μM (4), Pravastatin (20 μM (5), or Atorvastatin (10 μM) and L-mevalonate (100 μM) (6). GAPDH was used as a control for RNA loading. Quantification of RPA blots is expressed as the ratio of DR-α/GAPDH and CITTA/GAPDH signal for each sample. Similar results were obtained in independent experiments with ECs from four different donors. * p<0.001, ** p<0.02 compared to IFN-γ treated cells (2), *** p<0.001 compared to IFN-γ/Atorvastatin treated cells (3).

FIG. 3 is a comparison of two different functional consequences of inhibition of MHC class II antigens by statins on T lymphocyte activation.

the first consequence is shown by means of the histogram representing [³H]Thymidine incorporation measured in allogenic T lymphocytes exposed (5 days) to human ECs (solid bars) or human Mφ (open bars) or pretreated during 48 hrs with IFN-γ (500 U/mL) alone (1,3). or IFN-γ (500 U/mL) with Atorvastatine (10 μM) (2,4). Similar results were obtained in independent experiments with Mφ or ECs form three different donors. *p<0,02 compared to IFN-γ treated cells.

the second consequence is shown by means of the histogram representing IL2 release measured by ELISA in supernatants of allogenic T lymphocytes exposed (48 hrs) to human ECs (solid bars) or Mφ (open bars) pretreated 48 hrs with IFN-γ (500 U/mL) alone (1,3), or IFN-γ (500 U/mL) with Atorvastatin (10 μM) (2,4). Similar results were obtained in independent experiments with Mφ or ECs from four different donors. **p<0,01 compared to IFN-γ treated cells.

FIG. 4 is a combination of a graph and an electrophoretic gel showing that statins specifically decreased the expression of promoter IV of the transactivator CIITA on a transcriptional level.

FIG. 4a is a reproduction of an RNAse protection assay (RPA) for exon 1 of the promoter IV-specific form of CIITA (pIV-CIITA). Human vascular endothelial cells (ECs) unstimulated (1), treated with IFN-γ (500 U/ml, 12 hrs) alone (2), or with Atorvastatin (10 μM) (3), Lovastatin (10 μM) (4), Pravastatin (20 μM) (5), or Atorvastatin (10 μM) and L-mevalonate (100 μM) (6). GAPDH was used as a control for RNA loading. Quantification of RPA blots is expressed as the ratio of pIV-CIITA/GAPDH signal for each sample. Similar results were obtained in independent experiments with ECs from three different donors. * p<0.001, ** p<0.02 compared to IFN-γ treated cells (2), ** p<0.001 compared to IFN-γ/Atorvastatin treated cells (3).

FIG. 4b is a graph representing a densitometric analysis of RPA from actinomycin D (Act D) studies showing the effects of Atorvastatin on pIV-CIITA mRNA levels. ECs were pretreated with IFN-γ (500 U/ml, 12 hrs), and then Act D (10 μg/ml) was added alone or with Atorvastatin (10 μM) and RNA analyzed at different time points. Band intensities of pIV-CIITA/GAPDH mRNA ratio were plotted as a semi-log function of time (hours). Data represent mean ±SEM of separate experiments with cells from three different donors.

FIG. 4c is a blot representing a Western blots analysis (40 μg protein/lane) of ECs treated with IFN-γ (500U/ml) in the absence or presence of Lovastatin (10 μM) (Lova). Samples were analyzed for the phosphorylated form of Stat1-α (p Stat1-α) at different periods of time (minutes). Actin was used as a control for protein loading. Blots are representative of different experiments obtained with cells from four different donors.

FIG. 5 is a representation of the chemical structure of some commercially available statins.

FIG. 5a is a chemical representation of Atorvastatin and Lovastatin.

FIG. 5b is a chemical representation of Pravastatin sodium and Fluvastatin.

FIG. 5c is a chemical representation of Mevastatin and Simvastatin.

FIG. 6 is the association of a Western Blot and its graphic representation showing that Statins reduce IFN-γ induced CD40 expression on human atheroma-associated cells.

Western blot analysis for CD40 (1-8). Human vascular endothelial cells (ECs) under unstimulated conditions (1), treated with INF-γ (500 U/ml, 24 hrs) alone (2), or with Pravastatin (5 μM, 3), or with Lovastatin (5 μM, 4), or with, Atorvastatin (5 μM, 5), or with Simvastatin (5 μM 6), or with Simvastatin (10 μM) and L-mevalonate (200 μM) (7), Raji under unstimulated condition as positive control (8). Similar results were obtained in independent experiments with ECs from three different donors.

FIG. 7 is a Western Blot showing that Atorvastatin decreases IFN-γ induced CD40 protein expression on human atheroma-associated cells in a dose-dependant manner.

Western blot analysis for CD40 (1-6). Human vascular endothelial cells (ECs) under unstimulated conditions (1), treated with IFN-γ (500 U/ml, 24 hrs) alone (2), or with Aorvastatin, 5 μM (3), 2 μM (4), 0.4 μM (5), 0.08 μM (6). Similar results were obtained in independent experiments with ECs from three different donors.

FIG. 8 is a series of graph panels showing the functional effect of Statins on CD40 mediated pathways.

a, MCP-1 release measured by ELISA in supernatants of ECs exposed (24 hrs) with normal media (1), CD40L (5 μg/ml) alone (2), or with Pravastatin (5 μg) (3), or with Lovastatin (5 μM) (4), or with Atorvastatin (5 μM) (5), or with Simvastatin (5 μM) (6), or with simvastatin (5 μM) and L-Mevalonate (200 μM) (7). Similar results were obtained in independent experiments with ECs from four different donors. * p <0.05 3-6 compared to 2, and 7 compared to 6.

b, II-6 release measured by ELISA in supernatants of ECs exposed (24 hrs) with normal media (1), CD40L (5 μg/ml) (2), or with Pravastatin (5 μg) (3), or with Lovastatin (5 μM) (4), or with Atorvastatin (5 μM) (5), or with Simvastatin (5 μM) (6). Similar results were obtained in independent experiments with ECs from four different donors.* p <0.05 3-5 compared to 2, and 6 compared to 5.

c, II-8 release measured by ELISA in supernatants of ECs exposed (24 hrs) with normal media (1), CD40L (5 μg/ml) (2), or with Pravastatin (5 ;μg) (3), or with Lovastatin (5 μM) (4), or with Atorvastatin (5 μM) (5), or with Simvastatin (5 μM) (6). Similar results were obtained in independent experiments with ECs from four different donors. * p<0.05 3-5 compared to 2, and 6 compared to 5.

FIG. 9 is the association of immunostaining and its graphic representation showing that statins reduce CD40 and CD40L expression on human carotid atheroma.

A bank of human carotid atheroma from patients was analysed by immunostaining for CD40 and CD40L expression (FIG. 9B), 15 patients being treated with a statin for more than 3 months, 13 patients being not treated with. The statins are simvastatine or atorvastatine, at doses comprised between 20 and 40 mg par day.

FIG. 9A shows the graphical representation of CD40 staining area for the two groups;

FIG. 9C shows the graphical representation of CD40L staining area for the two groups

FIG. 10 is a graph showing the effect of Statins on mouse skin graft.

Mouse skin graft are harvested from the back region (˜2 cm²) of the animal and transplanted in the same back area of the recipient mice. Skin graft transplantation was analysed at day 7, 10 and 14 after the procedure.

Mice were treated with a given Statin (Atorvastatin) within oral food at the following daily doses: 1 mg/kg (low) or 100 mg/kg (high). Mice treated with normal food served as controls. At day 10 and 14 after transplantation, rejection is defined and measured in mice (granulation tissue and vascularisation) at the site where the graft were placed, using a Laser Doppler Perfusion Image (LDPI) system (Lisca, Inc).

FIG. 11 is a graph showing that Statin treatment reduces clinical score of collagen-induced arthritis.

From the day of first immunization with collagen, mice were treated with a given Statin (Atorvastatin) within oral food at the following daily doses: 1 mg/kg (low) or 100 mg/kg (high). Mice treated with normal food served as controls. There were 15 mice per group. One mice died after the first immunization (day 2) in the control group.

Shown is the clinical scores over 6 and 10 days of classical collagen-induced arthritis.

*=p<0.05

FIG. 12 is a table showing that Statin treatment suppresses collagen-specific T-lymphocyte response.

From the day of first immunization with collagen, mice were treated with a given Statin (Atorvastatin) within oral food at the following daily doses: 1 mg/kg (low) or 100 mg/kg (high). Mice treated with normal food served as controls. There were 15 mice per group. One mice died after the first immunization (day 2) in the control group.

At day 15 following the first immunization, mice were sacrificed and inguinal lymphocytes were cultured in the presence of collagen After 72 hours, T-lymphocytes proliferation and IFN-γ release were measured. Results are the mean +/− SD of four individual mice per treatment group, each of them tested in triplicate.

*=p<0.05

### DETAILED DESCRIPTION OF EMBODIMENTS OF INVENTION

In the context of the present invention, the following terms are defined in the following manner.

A statin is a molecule capable of acting as an inhibitor of HMG-CoA reductase. Members of the statin family include both naturally occurring and synthetic molecules, for example Compactin, Atorvastatin, Pravastatin, Lovastatin, Fluvastatin, Mevastatin, Cerivastatin, Simvastatin, Rosuvastatin. This list is not restrictive and new molecules belonging to this large family are regularly discovered. A statin may be hydrophilic, like Pravastatin, or lipophilic like Atorvastatin. Lipophilic statins are believed to better penetrate the tissues.

As discovered in the framework of the present invention, these molecules also have a second function, which is the capacity to inhibit IFN-γ-induced CIITA expression in appropriate cells. A conventional test for determining whether a given molecule is a statin or not is the inhibition of sterol synthesis, especially according to the analyzed tissues and cells^{19,20}.

A molecule which is <<chemically related or structurally equivalent>> to a statin refers to a molecule whose structure differs from that of any member of the statin family by 2 or less substitutions or by modification of chemical bonds. Examples of the structure of some statins are given in FIG. 5. Molecules which are chemically related or structurally equivalent to a statin, in accordance with the inventors, possess at leas the second above-mentioned function, which is the capacity to inhibit IFN-γ-induced CIITA expression in appropriate cells. This capacity is tested using the functional assay described below in the examples.

A molecule which is <<functionally equivalent>> to a statin refers to a molecule capable of measurable HMG-CoA reductase inhibitions. Thus at least all the molecules capable of competitively inhibiting the enzyme HMG-CoA reductase and called statins possess the required property. In addition, according to the inventors, the functionally equivalent molecules also possess the capacity to inhibit IFN-γ-induced CIITA expression in appropriate cells. Again, this capacity is tested using the functional assay described below in the examples. A molecule which is <<functionally equivalent>> to a statin may have a clinically insignificant lipid-lowering effect whilst having a clinically significant immunosuppressive effect. The lipid-lowering effect of a statin can be measured using conventional assays ^{19,20}. The term “compound” as used herein embraces statins and structural and functional equivalents thereof.

An IFN-γ responsive cell is a cell having a receptor in its membrane for IFN-γ and capable of transducing a signal after binding of IFN-γ. Some cells can be induced to express MHC class II by IFN-γ. The expression of MHC class II genes is considered a secondary response to INF-γ since a long lag period is required (24 hours for optimal response in some cases) and requires ongoing protein synthesis since cyloheximide and/or puromycin, agents that inhibit protein synthesis, abrogate IFN-γ-induced MHC class II expression.

MHC Class II molecules are heterodimeric glycoproteins that present antigen to CD4⁺ T cells, leading to T cell activation. Cells which are designated <<MHC class II positive>> express MHC class II molecules either constitutively or in response to stimulation, for example by IFN-γ, and have then MHC class II molecules inserted in their cellular membrane.

In the context of the therapeutic methods of the present invention, the following terms are defined in the following manner:

An immunomodulator is an agent whose action on the immune system leads to the immediate or delayed enhancement or reduction of the activity of at least one pathway involved in an immune response, whether this response is naturally occurring or artificially triggered, whether this response takes place as part of innate immune system or adaptive immune system or the both. An MHC Class II-mediated immunomodulator is an immunomodulator whose key action on the immune system involves molecules of MHC class II.

Immunomodulation is considered to be significant if for a given population of allogenic T-lymphocytes, T-cell proliferation is reduced or enhanced by at least 10% after exposure to a statin or functionally or structurally equivalent molecule, compared to the level of T-cell proliferation in the same individual without exposure to the same statin or same equivalent molecule. Whether or not the immunomodulation is significant can be tested using the functional assay described below.

An immunosuppressor is an agent which action on the immune system leads to the immediate or delayed reduction of the activity of at least one pathway involved in an immune response, whether this response is naturally occurring or artificially triggered, whether this response takes place as part of innate immune system or adaptive immune system or the both. An MHC Class II-mediated immunosuppressor is an immunosuppressor whose key action on the immune system involves molecules of MHC class II.

Immunosuppression is considered to be clinically significant if for a given population of T-lymphocytes, T-cell proliferation is reduced by at least 30%, and preferably at least 50%, after exposure to a statin or functionally or structurally equivalent molecule, compared to the level of T-cell proliferation in the same individual without exposure to the same statin or same equivalent molecule. Whether or not the immunosuppression is clinically significant can be tested using the following assay:

i) A sample of IFN-γ-responsive cells, such as monocytes-macrophases or endothelial cells, is recovered from a fist individual and divided into two batches, Batch 1 and Batch 2.

ii) Batch 1 of IFN-γ-responsive cells is treated for approximately 48 hours with IFN-γ (500 U/ml) alone. Batch 2 of IFN-γ-responsive cells is pre-treated for approximately 48 hours with IFN-γ (500 U/ml) and a statin or derivative (10 μM).

iii) Allogenic T-lymphocytes (for example, peripheral blood lymphocytes (<<PBL>>)) are recovered from a different donor, and exposed to pre-treated Batch 1 and Batch 2 of the IFN-γ-responsive cells (=co-incubation) for the appropriate time indicated below.

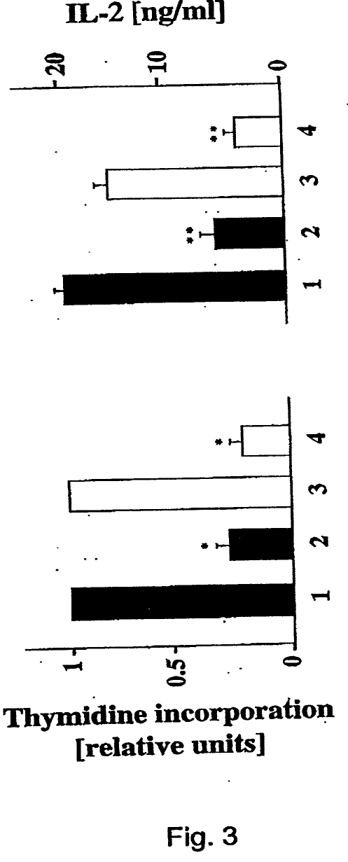

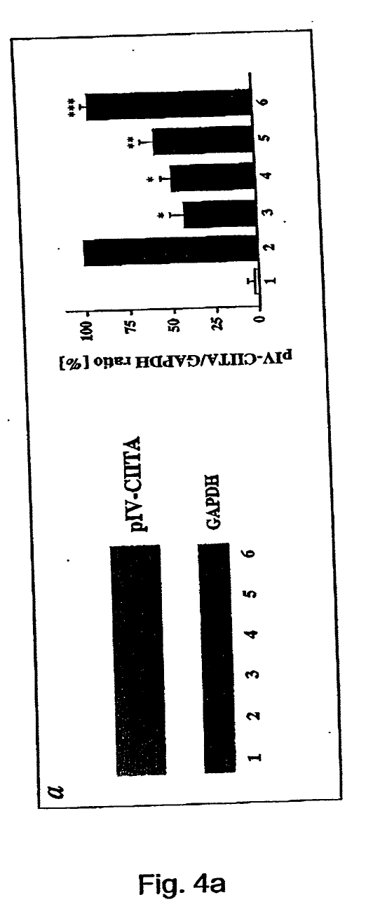

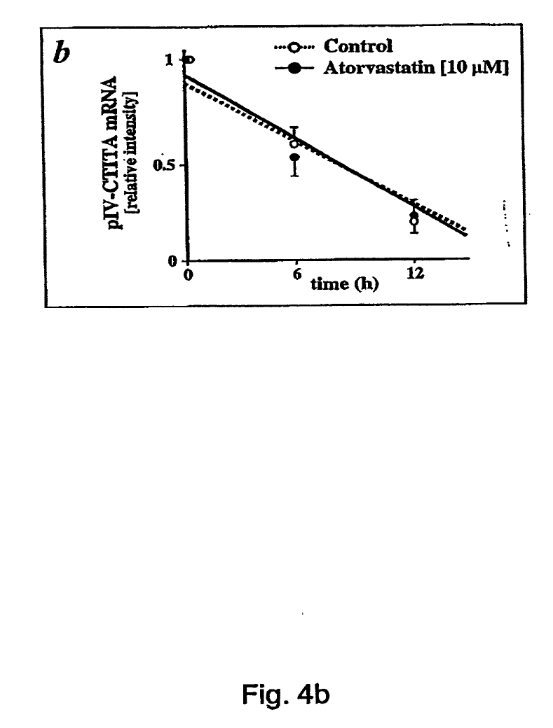

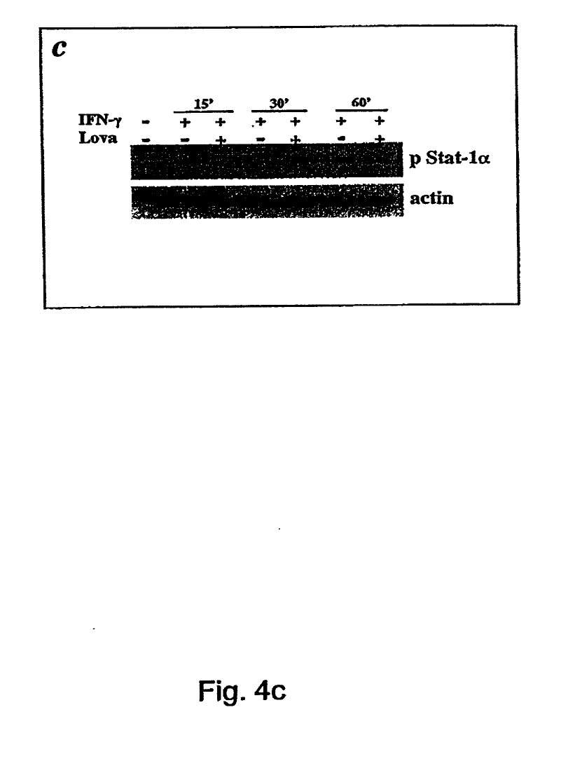

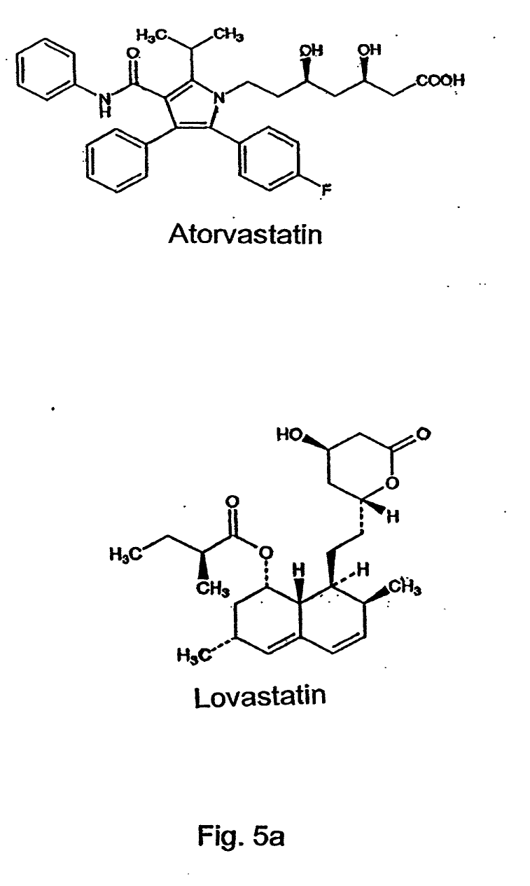

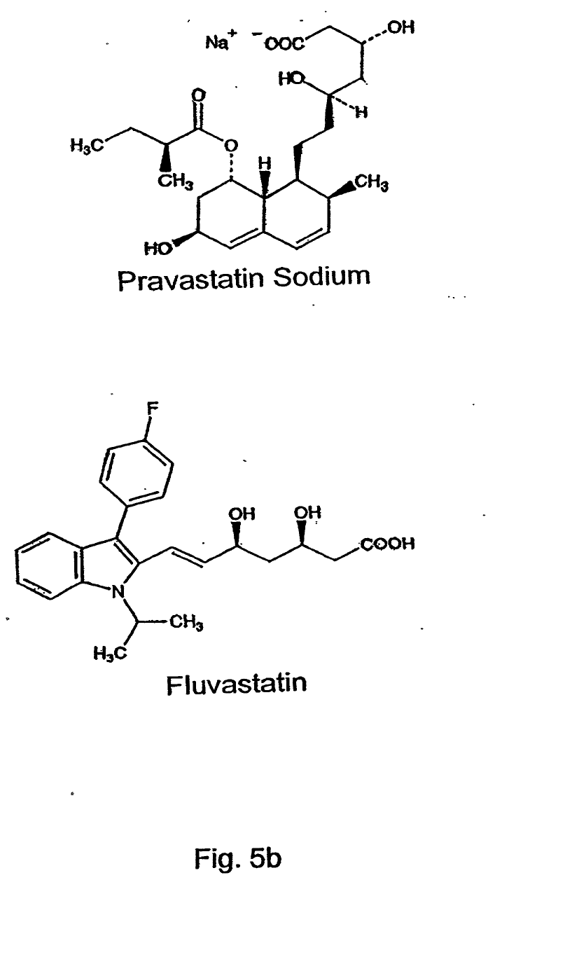

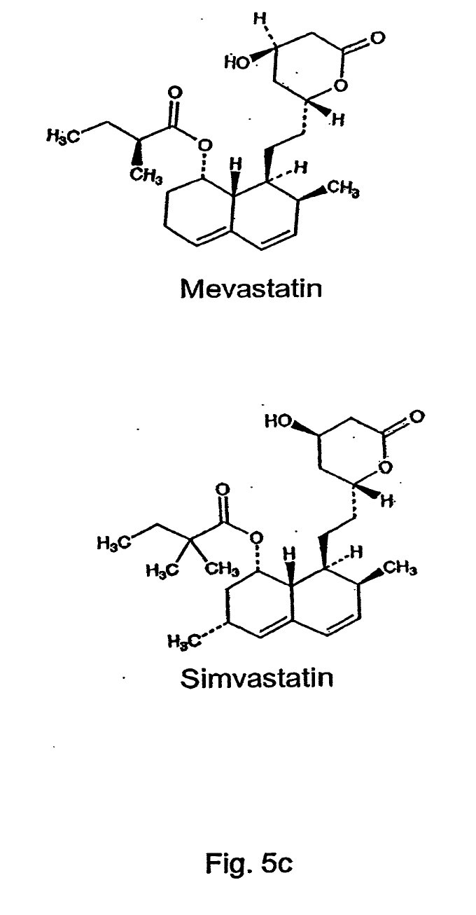

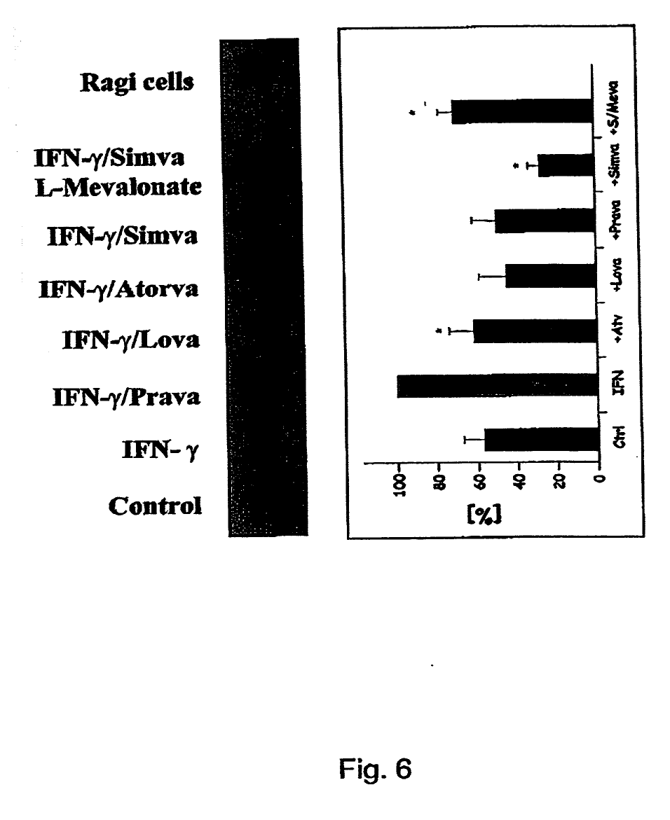

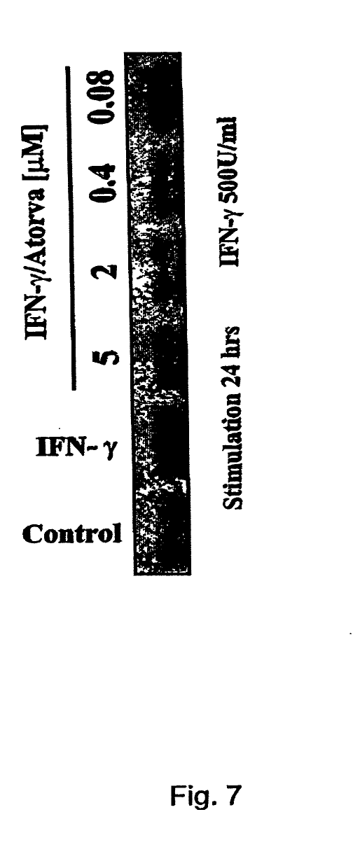

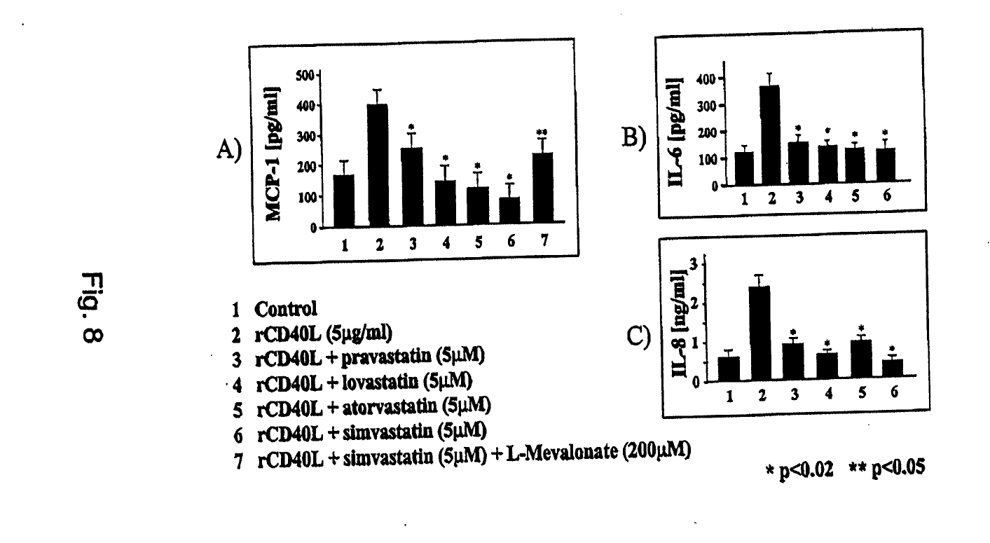

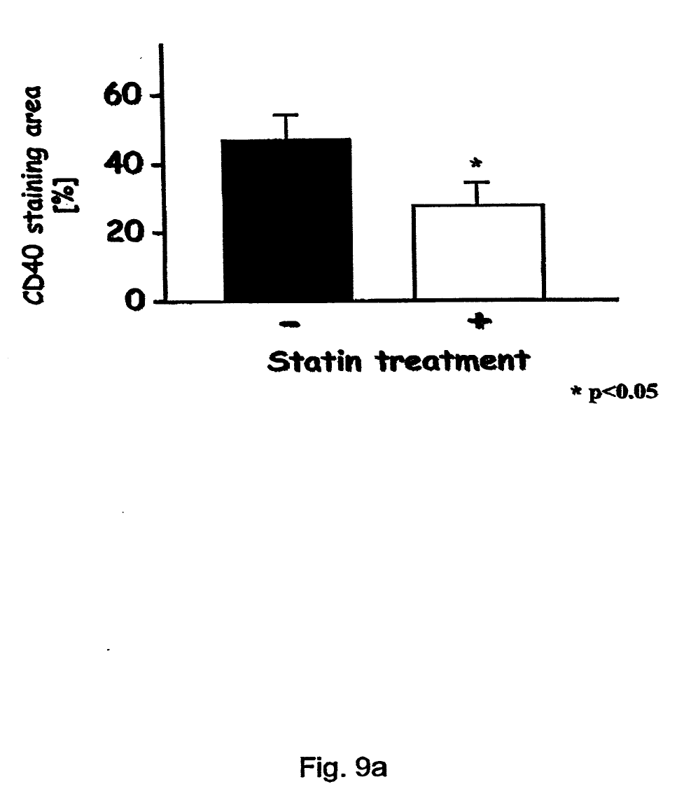

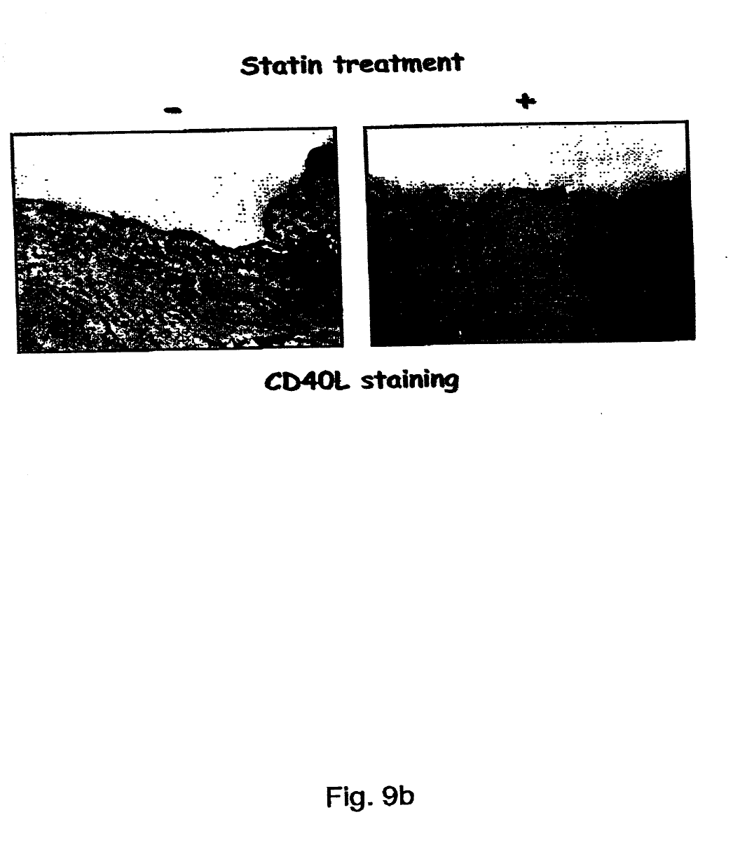

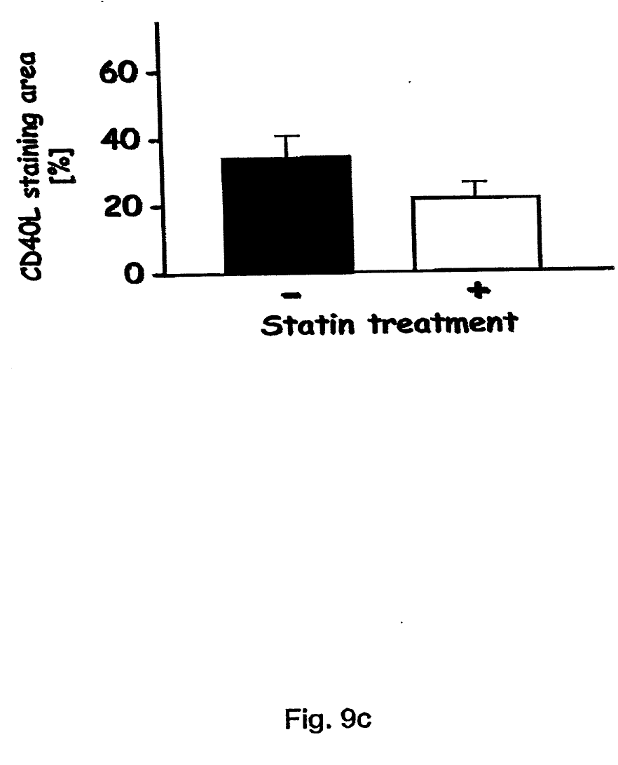

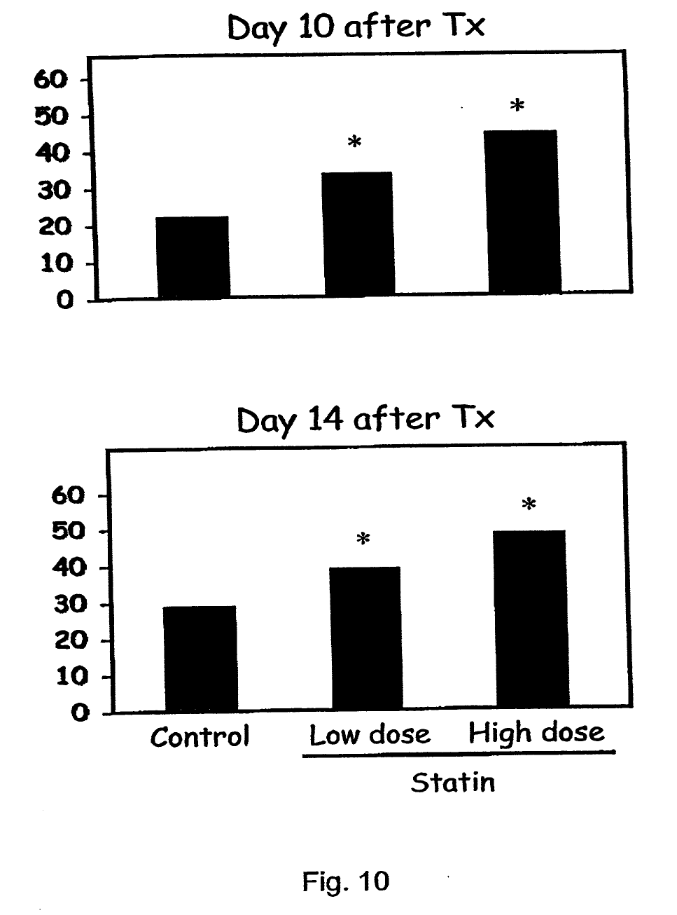

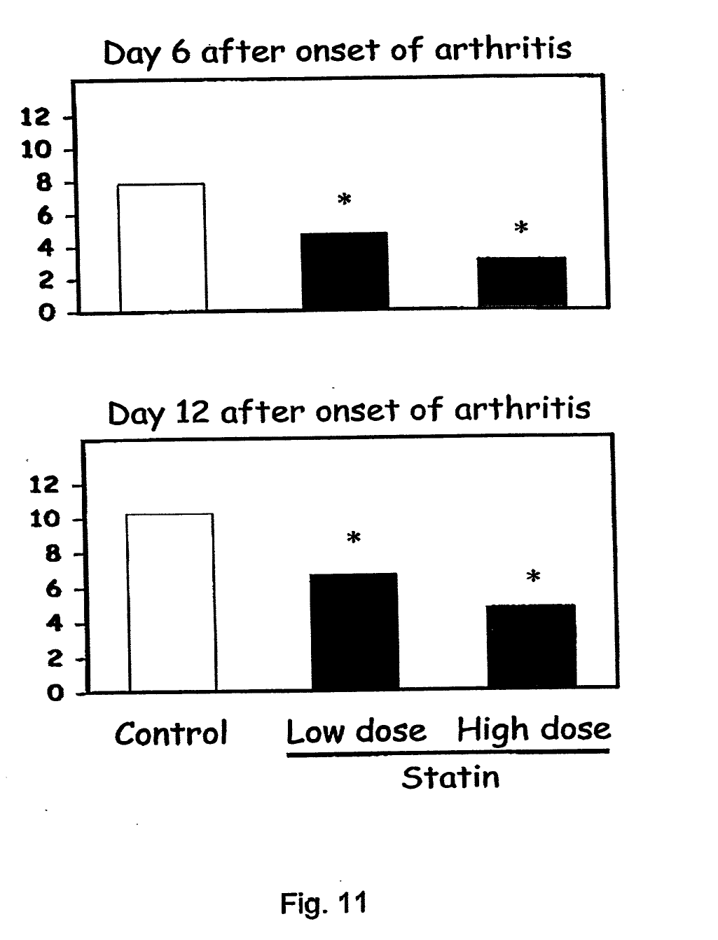

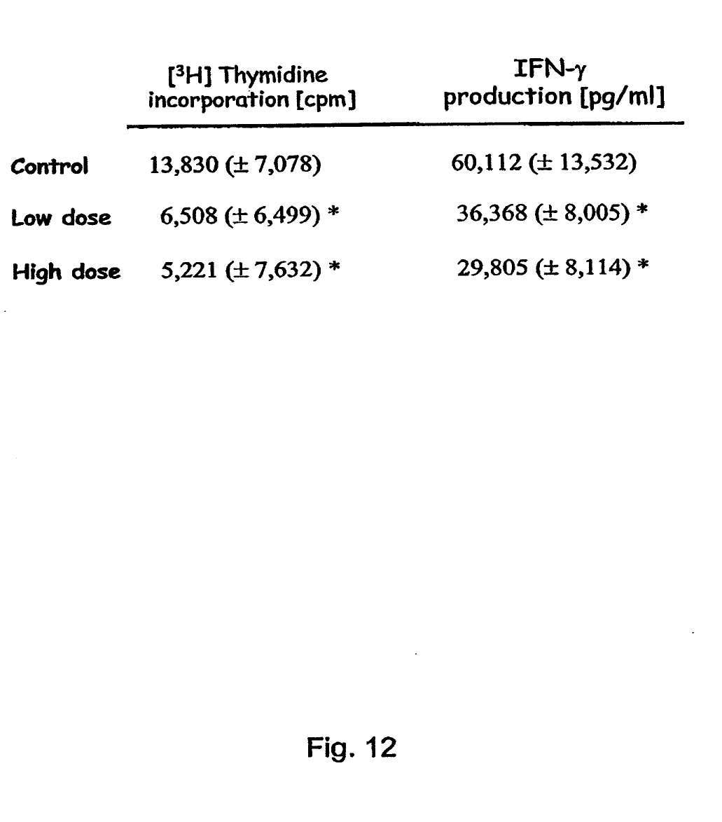

iv) [³H]Thymidine incorporation is measured during the last 24 hours of a 5-day co-incubation period as read-out for T-cell proliferation (see for example FIG. 3).

v) Or Interleukin-2 (IL-2) release is measured after a 2-day co-incubation period as read-out for T-cell proliferation (see for example FIG. 3).

vi) The read-out value for Batch 2 is expressed as a percentage of the read-out for Batch 1. If this value is equal to or less than 70%, preferably equal to or less than 50%, the statin or derivative is considered to have a clinically significant immunosuppressive effect.

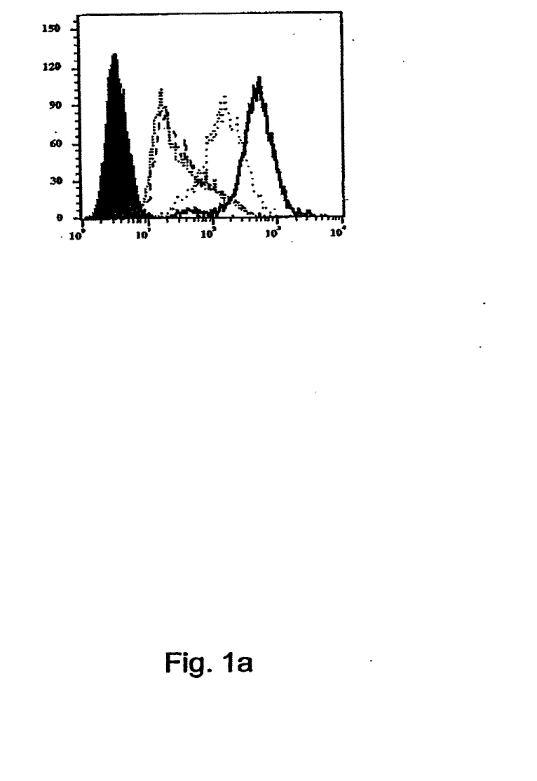

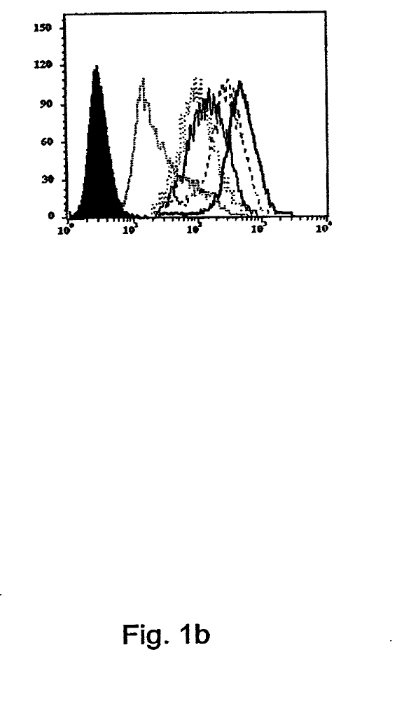

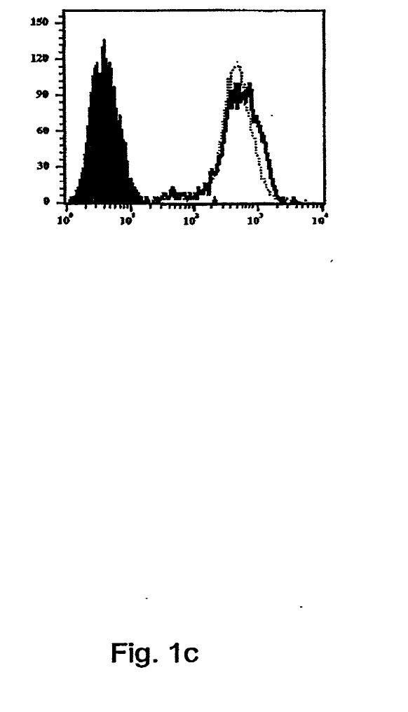

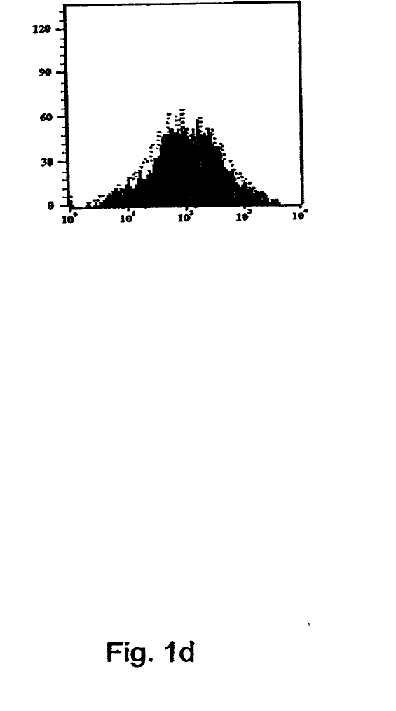

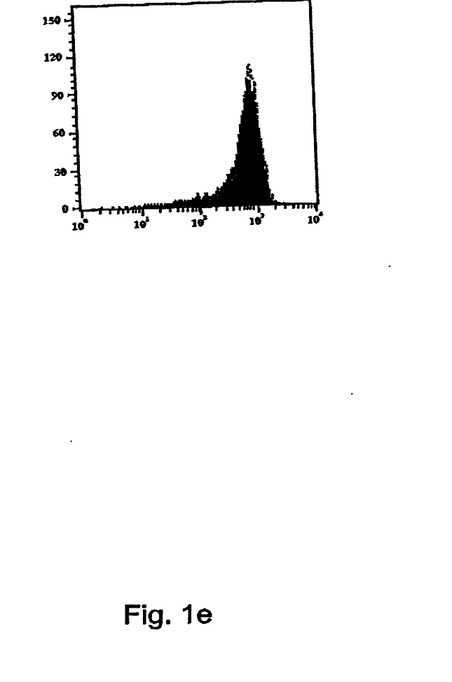

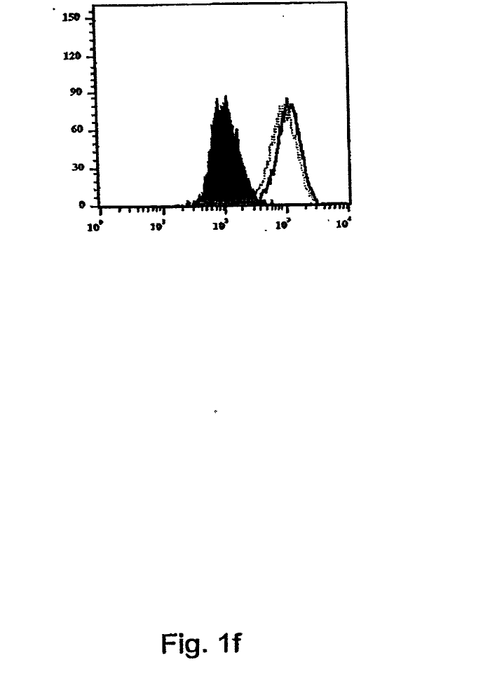

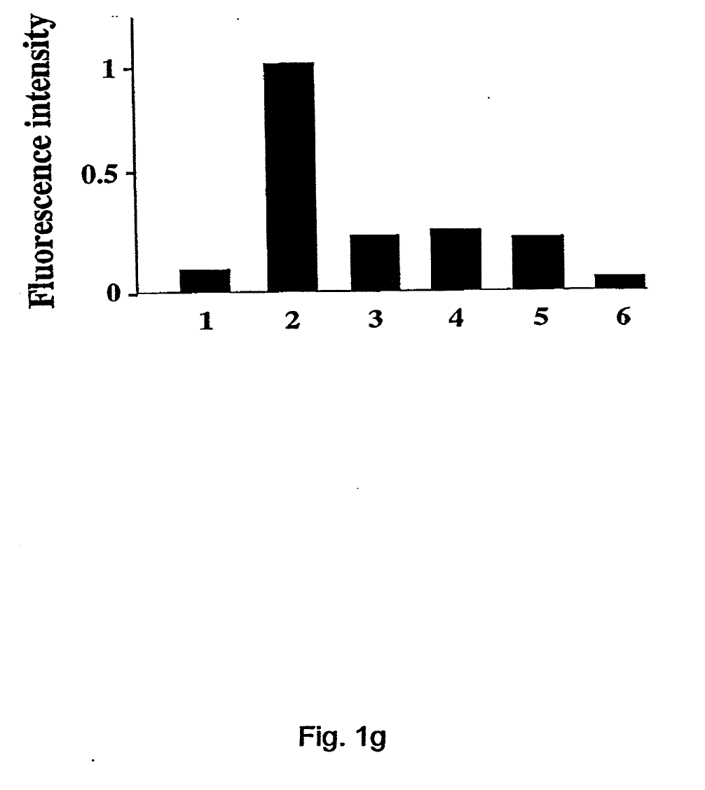

A further means of testing whether the immunosuppressive effect is clinically significant is to carry out the above assessment using Flow Cytometry (see for example FIG. 1).

An anti-inflammatory agent is an agent capable of reducing or inhibiting, partially or totally, immediately or after a delay, inflammation or one of its manifestations, for example migration of leucocytes by chemotaxis. An MHC Class II-mediated anti-inflammatory agent is an anti-inflammatory agent whose key action on the immune system involves molecules of MHC class II.

An anti immuno-inflammatory agent is an agent capable of reducing or inhibiting, partially or totally, immediately or after a delay, inflammation or one of its manifestations as well as other immune responses.

A detrimental immune response is an immune response which is painful or prejudicial to the health of a patient on a long or short-term basis. Immune reactions against self molecules or tissues, or against xenografted tissues or organs are examples of detrimental immune responses.

Immunosuppression (or immunomodulation) becomes clinically desirable in cases where the immune system acts detrimentally to the health of a patient or is feared to do so, the shut down or down-regulation of the immune response being then considered as useful by the physician for the health of the patient. Such conditions can be encountered after an organ transplantation for enhancing tolerance to the Another example is autoimmune disease, including type I diabetes, multiple sclerosis and rheumatoid arthritis. Cases in which immunosuppression is clinically required are not limited to those cited but further include psoriasis and other pathologies. Moreover, immunosuppression also includes prevention of undesirable immune reactions, for example before transplantation.

A transplantation concerns organ or tissue, such as heat, kidney or skin.

A first aspect of the invention involves the exploitation of the molecular implication of statins and their structural and functional equivalents in IFN-γ-mediated cell responses.

According to one embodiment of this first aspect, statin, for example, can be used in a process to regulate the IFN-γ-induced CIITA expression in IFN-γ responsive cells. This process is implemented by contacting an IFN-γ responsive cell with at least one statin. A consequence of this regulation is the possibility to regulate CIITA-dependant intra- and intercellular events. The role of CIITA being crucial in the cell, particularly for the expression of MHC class II molecules, acting on this important transactivator is a unique way to interfere with MHC class II transcription, expression and thus presentation to T lymphocytes. Similarly, repression of CIITA expression leads to the repression of T lymphocyte activation and proliferation. This leads in turn, at least partially, to the inhibition of all depending intercellular events characterizing the complex cascade of the immune response.

The process described above can be cared out either in vow or in vitro.

For this process of regulation of IFN-γ-induced CIITA expression, molecules other than statins can be used provided they are chemically related to at least one statin and/or functionally equivalent thereto. In a preferred embodiment, the statins are used and the used statin is Compactin, Atorvastatin, Lovastatin, Pravastatin, Fluvastatin, Mevastatin, Cerivastatin, Rosuvatin or Simvastatin. In a particularly preferred embodiment, especially when treating a patient in preparation for or after organ or tissue transplant, the used statins is Compactin, Atorvastatin, Lovastatin, Fluvastatin, Mevastatin, Cerivastatin or Simvastatin,

Among IFN-γ responsive cells are cells which become APC (Antigen Presenting Cells) upon induction by IFN-γ. These particular cells, called <<facultative APCs>>, are able to become MHC class II positive i.e. displaying MHC class II molecules on their surface if suitably stimulated. Such cells can be primary human endothelial cells, primary human smooth muscle cells, fibroblasts, monocytes-macrophages, cells of the central nervous system, ThP1, melanomas or Hela cells.

As the statins' action on stimulated CIITA expression is both dose-dependant and dependant of the type of statin, this process of contacting a cell with a particular member of the statin family at a particular dose provides a useful opportunity to control quantitatively the CIITA-expression and to set it at a given level. The relation between CIITA expression and level of MHC class II mRNA being linear, this quantitative control over expression of CIITA is transposable to MHC class II transcription and translation, i.e. MHC class II expression.

In the process of regulation of IFN-γ-induced CIITA expression described above, the regulation of IFN-γ-induced CIITA expression is preferably an inhibition or a reduction of this expression.

In a preferred mode of action of statins, or functional or structural derivatives, the regulation of IFN-γ-induced CIITA expression is solely achieved by inhibition of the CIITA inducible promoter IV. By “solely achieved” is meant that the statins have no effect, or substantially no effect, on the constitutive expression of CIITA, namely expression regulated by promoters I and III¹¹.

As mentioned above, it is surprisingly the effect of statins as HMG-CoA reductase inhibitors that mediates repression of MHC class II by inhibition of CIITA. Indeed providing the cell with L-mevalonate, which is the product of HMG-CoA reductase, abolishes inhibition by statins. The process of the invention has thus the property that the regulation is reversible at least partially, and preferably fully, by addition of L-mevalonate.

According to a further embodiment of this first aspect, the invention also concerns a screening method, more particularly a method for identifying molecules capable of inhibiting IFN-γ-induced CIITA expression, this inhibition being at least partially reassemble by addition of L-mevalonate. This method is carried out by contacting a cell which is IFN-γ responsive, with a candidate inhibitory molecule and with IFN-γ. In a second step of the method, inhibition or absence of MHC class II expression in presence of the candidate molecule is detected. The next step is to contact the cell with L-mevalonate and to detect a total or partial reversal of the inhibitory effect.

Inhibition of IFN-γ induced CIITA expression at least partially by acting on the HMG-CoA reductase is an unexpected effect with significant clinical potential; molecules capable of effecting this can be identified by screening as described. The tested property is the ability to inhibit IFN-γ induced CIITA expression in at least partially reversible manner by addition of L-mevalonate.

The detection can be made on the basis of MHC class-II expression or directly by CIITA expression. For detection of MHC class-II expression, the cells used must be responsive to stimulation by IFN-γ, preferred cells for this purpose are endothelial cells. IFN-γ and the potential inhibitor molecule are contacted with the cells; the detection of MHC class II expression is then carried out. In particular, this step can be accomplished by incubating the cells with for example fluorophore-conjugated specific antibody and then testing by flow cytometry. The skilled man will be aware of other classical ways to detect MHC-class II expression, for example by performing mixed lymphocytes reaction (allogenic T lymphocytes incubated with IFN-γ and candidate molecule-pretreated human endothelial cells) and assaying T cell proliferation.

A second possibility is to use a direct screen for inhibition of the CIITA promoter IV activity by employing transfectants containing a reporter gene under the control of CIITA promoter IV (see for example reference 9).

If the candidate molecule appears to be an efficient inhibitor, the additional property of reversibility is tested in a further step which comprises the addition of L-mevalonate to the previous cell culture and detection of a total or partial reversal of the inhibitory effect. This means that expression of MHC class II molecules is at lot partially restored. Methods to assay this expression are the same as above. This method also provides a test for identifying functional equivalents of statins.

Implementation of this screening method leads to the selection of inhibitors of CIITA expression which can be then used as such. Following the mode of selection, their action on CIITA is at least partially reversible by addition of L-mevalonate. Inhibitors found according to this screening method may be useful as medicaments having immunosuppressive and anti-inflammatory effects or for example in fundamental biology to determine how L-mevalonate derivatives interfere in stimulation by interferon γ.

A second aspect of the invention concerns therapeutic methods exploiting the effects of statins. The novel effect of statins as an effective MHC class II repressor and more particularly the mechanism of this effect via repression of promoter IV of the MHC-II transactivator CIITA provides a firm scientific rationale for the use of this drug as an immunosuppressor in organ transplantation. It also suggests numerous other practical clinical applications of statins as novel immunomodulators, in particular in diseases where aberrant expression of MHC class II and/or aberrant activation of CD4 T lymphocytes are implicated. Beyond organ transplantation, this ranges from various autoimmune diseases (including type I diabetes, multiple sclerosis and rheumatoid arthritis) to conditions such as psoriasis and chronic inflammatory diseases such as atherosclerosis. The fact that statins are well-tolerated drugs may qualify them as a welcome addition to the limited current arsenal of immunosuppressive agents.

Specifically, in a first embodiment, the invention concerns a method to achieve immunomodulation in a subject in need of such treatment, this immunomodulation being mediated via MHC class II. A subject, for example a mammal, is likely to be treated by this method if he is suffering from a condition involving inappropriate immune response or if he is susceptible of suffering from it. The method comprises administering to the subject at least one statin or a functionally or structurally equivalent molecule, in an amount effective to modulate MHC class II expression in the subject. The modulation may begin to occur immediately on administration of the statin, or may become effective within a few hours, e.g. 8 to 48 hours of administration.

In a second embodiment, the invention concerns a method to achieve immunosuppression in a mammal in need of such treatment, this immunosuppression being mediated via the MHC class II. In a preferred variant the repression is the result of repression of T lymphocyte activation. A mammal is likely to be treated by this second method if he is suffering from a condition involving detrimental immune response or if he is susceptible to suffer from it. The method comprises administering to the mammal at least one statin, or a functionally or structurally equivalent molecule, in an amount effective to suppress MHC class II expression in the subject. The suppression may begin to occur immediately on administration of the statin, or may become effective within a few hours, e.g. 8 to 48 hours of administration.

In a third embodiment, the invention concerns a method exploiting the major role of MHC class II expression in inflammation process in general i.e. a method to achieve MHC-class II mediated anti-inflammatory effect in a mammal in need of such treatment. A mammal is likely to be treated by this second method if he is suffering from a condition involving detrimental immune response or if he is susceptible to suffer from it. The method comprises administering to the mammal at least one statin, or a functionally or structurally equivalent molecule, in an amount effective to suppress MHC class II expression in the subject.

In a fourth embodiment, the invention concerns a method to achieve CD40mediated anti immuno-inflammatory effect in a mammal in need of such treatment. The method comprises administering to the mammal at least one statin, or a functionally or structurally equivalent molecule, in an amount effective to modulate CD40 expression, in particular the inducible expression of CD40, most preferably the IFN-γ induced CD40 expression.

The subject treated by anyone of the four mentioned methods is preferably a human. The following properties or applications of these methods will essentially be described for humans although they may also be applied to non-human mammals, for example apes, monkeys, dogs, mice, etc. The invention therefore can also be used in a veterinarian context.

A patient population susceptible of being treated by methods of the present invention includes patients who in addition to suffering from a condition involving inappropriate or detrimental immune response, may also suffer from hypercholestemlaemia, or from problems in the metabolism of lipids, particularly LDL (low-density lipoproteins), involving high levels of certain lipids. A particularly preferred group of subjects likely to be treated by one of the three methods is a subject who does not suffer from hypecholesterolaemia, irrespective of whether he has or not other risk factors for heart disease and stroke. By hypercholesterolaemia, it is meant LDL-cholesterol levels above 220 mg/dL, preferably above 190 mg/mL, after diet In cases where a patient presents risk factors for heart disease or stroke, the ‘threshold’ level beyond which hypercholesterolacmia is considered to occur can be lower, for example down to 160 mg/dL, even down to 130 mg/dL.

The inhibition by statins of MHC class II expression is specific for IFN-γ-induced condition. This specificity is very advantageous since the immune system as a whole is not disturbed by statin. This characteristic of the treatment of the invention is of great interest since the patient under treatment is still able to fight opportunistic infections.

The methods arm particularly well suited when the subject is suffering from a condition which involves IFN-γ inducible CIITA expression. Some autoimmune diseases are known to involve inappropriate IFN-γ release leading to CIITA expression in cells which do not normally express CIITA. It is for this reason that autoimmune diseases in general are particularly preferred conditions from which the subject is suffering.

Diseases which can be considered as autoimmune, are numerous. The described methods of the invention (i.e. immunomodulation, immunosuppression and regulation of inflammation) are particularly susceptible to be effective on type I diabetes, multiple sclerosis, rheumatoid arthritis, Crohne's disease and Lupus erythematosus.

Another appropriate application of one of the described methods, but particularly the immunosuppressive one, is that arising from an organ or tissue transplantation. In such an operation, the total immunological compatability between the subject (i.e. the graft recipient) and the graft donor is almost impossible unless it is an autograft. Cells of the recipient, detecting the presence of non-self cells, are likely to kill those cells leading to the rejection of the graft. Improvement of the tolerance of the recipient is needed and can be accomplished by means of the immunosuppressive method described above.

In particular, statin treatment is well suited to skin transplantation. The need for skin graft arises for example from skin ulcers. Skin ulcer treatment generally includes the Organogenesis system of Appligraft©; but this system suffers from allo-rejection. Co-treatment with statin according to the invention is thus an example of application of the present invention.

Statin treatment can be used in connection with implantable biological prostheses, for example with resilient, biocompatible two or more layered tissue prosthesis which can be engineered into a variety of shapes and used to repair, augment, or replace mammalian tissues and organs. Statin treatment reduces or suppresses inflammation and immune rejection at the site of implantation, the prosthesis thus undergoes controlled biodegradation accompanied by adequate living cell replacement, or neo-tissue formation, such that the original implanted prosthesis is remodeled by the host's cells before it is degraded by host enzymes.

The methods of the invention can be used in a preventive manner if a detrimental immune response is likely to arise. This is particularly convenient in the case of transplantation where the detrimental immune response is known to be triggered by the graft. Increased tolerance must be achieved before the transplantation and is an important part of the operation.

Other conditions which way be treated by the methods of the invention are psoriasis and inflammation in general or chronic inflammatory diseases, such as atherosclerosis.

The methods of the invention are particularly well suited for a topical application, for example in dermatology. The topical delivery of statin for example on skin or eye, is very useful to achieve high local concentrations without side effects. The application can be localized directly on the site of inflammation. This way of administering statin is useful in the local treatment of psoriasis, eczema and other skin inflammation. This is also useful for treatment of eye inflammation like uveitis.

For this type of application, the statins, or their structural or functional derivatives, are administered in the form of a cream, a spray, a lotion, an ointment, a powder or a needle-less injection, where the inflammation occurs.

The statin used to carry out one of the methods as described above is preferably Compactin, Atorvastatin, Lovastatin, Pravastatin, Fluvastatin, Mevastatin, Cerivastatin, Rosuvastatin or Simvastatin. The preferred statins for the present invention are those having lipophilic properties. Due to their lipophilic properties, in the case of organ or tissue transplants, the preferred statins are Compactin, Atorvastatin, Lovastatin, Fluvastatin, Mevastatin, Cerivasttin, or Simvastatin. The most preferred statin is Atorvastatin.

Since the lipid lowering effect of the currently used statins mentioned above can be, under certain circumstances, an inopportune effect, it would be advantageous in these circumstances to benefit firm an immunomodulatory, immunosuppressive or anti-inflammatory effect of statins, without the lipid-lowering effect. In such cases, the methods of the invention are then preferably carried out with a statin, or a functional or structural derivative, exhibiting an immunomodulatory effect without a therapeutically significant lipid-lowering effect when administered at conventional doses. By “therapeutically significant,” it is understood that while such compounds can provide some amount of HMG-CoA reductase inhibition, even when measured in vitro, they are poor choices for use in the treatment of such conditions as hypercholesterolaemia or problems in the metabolism of lipids.

The methods can be part of a more general treatment of the subject or can be accompanied by a different treatment. In this case, the statin or derivative can be administered with or without other immunosuppressive drugs. In cases where other immunosuppressive drugs are administered, the immunosuppressive drugs may be administered separately, simultaneously or sequentially. In a particular case, the statin is administered in the absence of any other immunosuppressive agents, the statin is not administered in combination with cyclosporin A or cyclophosphamide.

In each method, depending on the chosen statin, or structurally or functionally equivalent derivative, the amount given to the subject must be appropriate, particularly effective to specifically modulate IFN-γ inducible MHC class II expression.

As for every drug, the dosage is an important part of the success of the treatment and the health of the patient. The degree of efficiency as immunomodulator, immunosppresor or anti-inflammatory agent depends on the statin or derivative used. An appropriate amount is comprised for example between about 1 and about 500 mg per day and more preferably 10 and 80 mg per day. Most preferably, when using a commercially available statin, between 20 and 40 mg per day for currently used statins. It is envisaged that more effective statins may be discovered in the future, these molecules will thus be administered to the subject in smaller quantities. In every case, in the specified range, the physician has to determine the best dosage for a given patient, according to his sex, age, weight, pathological state and other parameters.

In the context of the methods of the invention described herein, the administration mode comprises intralesional, intraperitoneal, intramuscular or intravenous injection; infusion; or topical, nasal, oral, ocular or otic delivery. While compounds may be administered continuously, a particularly convenient frequency for the administration of statin or derivative is once a day.

Since statins play a role in immune response, they can be used as immunosuppressors, immunomodulators or anti-inflammatory agents for the manufacture of a medicament for use in the treatment of a condition involving aberrant, undesirable or detrimental expression of MHC class II. Statins can be replaced by structurally or functionally equivalent molecules.

The present invention also concerns a method of treating a patient afflicted with an autoimmune disease, comprising administering to said patient a compound that inhibits 3-hydroxy-3-methylglutaryl coenzyme A (HMG-CoA) reductase in an amount effective to treat said disease. Preferred compounds are compounds having a therapeutically insignificant lipid-lowering effect and which suppress MHC Class II expression.

The present invention also concerns a method of treating a patient suffering from an autoimmune disease or condition comprising administering to said patient at least one compound, capable of measurable HMG-CoA reductase inhibition and inhibition of MHC Class II expression in said patient, in an amount effective to treat such autoimmune disease or condition.

The present invention also concern a method of treating a patient in preparation for or after an organ tissue transplant comprising administering to said patient at least one compound capable of measurable HMG-CoA reductase inhibition and inhibition of MHC Class II expression in said patient, in an amount which is effective to prevent tissue rejection.

The present invention also concerns a method of preventing or treating tissue or organ rejection in a patient comprising administering to said patient a compound that inhibits 3-hydroxy-3-methylglutaryl coenzyme A reductase (HMG-CoA reductase) in an amount effective to prevent or treat tissue or organ rejection.

The present invention also concerns a method of eating an autoimmune disease or an immuno-inflamnatory disease, comprising administration of at least one statin, or a functionally or structurally equivalent molecule, to a subject in an amount effective to modulate IFN-γ inducible MHC class II expression and/or CD40 expression in the subject, such that the symptoms of said disease at least partially alleviated. A particularly preferred disease is rheumatoid arthritis. A preferred subject does not suffer from hypercholesterolemia.

According to the present invention, for the treatment of rheumatoid arthritis, the statin is administered in conjunction with another rheumatoid arthritis therapy. Preferred rheumatoid arthritis therapies are selected from the group consisting of steroids; nonsteroidal anti-inflammatory agents; (NSAIDs); disease modifying anti-rheumatoid drugs (DMARDs); and combinations thereof.

Preferred nonsteroidal anti-inflammatory agents are selected from the group consisting of salicylates; fenoprofen; naproxen; piroxicam; tolmetin; indomethacin; sulindac; meclofenamate; and combinations thereof. Preferred disease modifying anti-rheumatoid drugs are selected from the group consisting of D-penicillamine; gold salts (both parenteral and oral forms); hydroxychloroquine; azathioprine; methotrexae; cyclophosphanide; and combinations thereof.

The present invention also concerns the use of a statin or a functionally or structurally equivalent molecule, for the preparation of a medicament for treating an autoimmune disease or an immuno-inflammatory disease, such statin being present in an amount effective modulate IFN-γ inducible MHC class II expression and/or CD40 expression, thereby alleviating at least partially the symptoms of said disease.

The present invention also concerns a method of preventing or treating tissue rejection in a subject comprising administering to said subject at least one statin or a functionally or structurally equivalent molecule in an amount which is effective to inhibit IFN-γ inducible MHC Class II expression and/or CD40 expression such that rejection is at least partially prevented or treated.

The present invention also concerns a method of treating a tissue graft prior to, during or after transplantation, comprising administering to a patient a statin or a functionally or structurally equivalent molecule, in an amount which is effective to inhibit IFN-γ inducible MHC Class II expression and/or CD40 expression effective such that inflammation or tissue rejection, or both, is reduced.

Preferred tissue grafts are tissue graft selected from the group consisting of skin; bone; abdominal wall; pericardium; periosteum; perichondrium; intervertebral disc; articular cartilage; dermis; epidermis; ligaments; bowel and tendons.

The present invention also concerns the use of a statin or a functionally or structurally equivalent molecule in the preparation of a medicament for reducing inflammation or for reducing tissue rejection, or both, such statin being present in an amount effective to inhibit IFN-γ inducible MHC Class II expression and/or CD40 expression such that inflammation or tissue rejection, or both, is reduced, for administration to a subject before, during or after a tissue graft.

The present invention also concerns a kit comprising a tissue graft material and a statin, or a functionally or structurally equivalent molecule, either in the same or separate packaging. For the kit, the tissue graft material is preferably selected from the group consisting of skin; bone; abdominal wall; pericardium; periosteum; perichondrin; intervertebral disc; articular cartilage; dermis; epidermis; bowel; ligaments; and tendons.

The present invention also concerns a method of preventing or treating organ rejection in a subject comprising administering to said subject prior to or during transplantation, at least one statin or a functionally or structurally equivalent molecule, in an amount which is effective to inhibit IFN-γ inducible MHC Class II expression and/or CD40 expression such that rejection is at least partially prevented or treated. Preferred organs are heart, kidney, and liver.

The present invention also concerns a method of treating an inflammatory disorder comprising administering to a subject, at least one statin or a functionally or structurally equivalent molecule, in an amount which is effective to inhibit IFN-γ inducible MHC Class II expression and/or CD40 expression such that inflammation is reduced. The inflammatory disorder is preferably selected from the group consisting of inflammatory skin disease, inflammatory ocular disorder, and lupus erythematosus.

The present invention also concerns the use of a statin or a functionally or structurally equivalent molecule in the preparation of a medicament for reducing inflammation in an inflammatory skin disorder, such statin being present in an amount effective for reducing inflammation.

In the context of the present invention, a preferred inflammatory disorder is an ocular disorder, in particular uveitis.

The present invention also concerns the use of a statin or a functionally or structurally equivalent molecule in the preparation of a medicament for reducing inflammation in an inflammatory ocular disorder, such statin being present in an amount effective for reducing inflammation.

### EXAMPLES

### Example 1

### Materials and Methods

Reagents, Human recombinant IFN-γ was obtained from Endogen (Cambridge, Mass.). The three statins used in these studies [Atorvastatin, (Parke Davis); Lovastatin (Merk Sharp and Dohme); and Pravastatin (Bristol-Myers Squibb)] are commercially available and were obtained from commercial sources. Mouse anti-human MHC class II and MHC class I fluorescein isothiocyanate-conjugated (FITC) and unconjugated monoclonal antibodies were purchased from Pharmingen (San Diego, Calif.). Cycloheximide, actinomycin and L-mevalonate were purchased from Sigma (St. Louis, Mo.).

Cell isolation and culture. Human vascular endothelial cells (ECs) were isolated from saphenous veins by collagenase treatment (Worthington Biochemicals, Freehold, N.J.), and cultured in dishes coated with gelatin (Difco, Liverpool England) as described elsewhere¹⁵. Cells were maintained in medium 199 (M199; BioWhittaker, Wokingham, England) supplemented with 100 U/ml penicillin/streptomycin (BioWhittaker), 5% FCS (Gibco, Basel, Switzerland), 100 μg/ml heparin (Sigma) and 50 μg/ml ECGF (endothelial cell growth factor, Pel-Freez Biological, Rogers, AK). Culture media and FCS contained less than 40 μg LPS/ml as determined by chromogenic Limulus amoebocyte-assay analysis (QLC-1000; BioWhittaker). Endothelial cells were >99% CD31 positive as characterized by flow cytometry and were used at passages 24 for all experiments.

Monocytes were isolated from freshly prepared human peripheral blood mononuclear cells obtained from leukopacs of healthy donors following Ficoll-Hypaque gradient and subsequent adherence to plastic culture flasks (90 min., 37° C.). Monocytes were cultured in RPMI 1640 medium (BioWhittaker) containing 10% FCS for 10 days¹⁵. Macrophages derived from monocytes were >98% CD64 positive as determined by flow cytometry.

The human Raji cell line (Epstein-Barr virus (EBV)-positive Burkitt lymphoma cell line) obtained from American Type Culture Collection (Rockville, Md.) and the human dendritic cells obtained as described¹⁶ were grown in RPMI-1640 medium containing 10% FCS.

Flow cytometry. Cells were incubated with FITC-conjugated specific antibody (60 min, 4° C.) and analyzed in a Becton Dickinson FACScan flow cytometer as described¹⁵. At least 100,000 viable cells were analyzed per condition. Data were analyzed using CELLQUEST software (Becton Dickinson).

Immunolabeling. Cells grown on coverslips were fixed for 5 min with methanol at −20° C. The coverslips were rinsed and incubated successively with 0.2% Triton X-100 in PBS for 1 hour, 0.5 M NH₄Cl in PBS for 15 min and PBS supplemented with 2% bovine serum albumin (Sigma) for another 30 min. Cells were then incubated overnight with primary antibody (1:200) in 10% normal goat serum (Sigma)/PBS. After rinsing, the coverslips were incubated with secondary antibodies FITC-conjugated (1:1000) for 4 h. All steps were performed at room temperature and in between incubation steps cells were rinsed with PBS. Cells were counterstained with 0.03% Evans blue/PBS. Coverslips were mounted on slides in Vectashield (Vector Laboratories, Burlingame, Calif.). Cells were examined using a Zeiss Axiophot microscope equipped with appropriate filters. Specificity of the immunolabeling was checked for by replacing the primary antibody with PBS.

RNAse protection assays. Total RNA was prepared with Tri reagent (MRC, Inc., Cincinnati, Ohio) according to the manufacturers instructions. RNAse protection assays with 15 μg of RNA per reaction were carried out as described previously¹² using human probes for MHC class II (DR-α, CIITA, exon 1 of the promoter IV-specific form of CIITA (pIV-CIITA), and GAPDH as a control for RNA loading. Signal quantitation was determined using a phosphoimager analysis system (Bio-Rad, Hercules, Calif.). Levels of DR-α, CIITA, and pIV-CIITA RNA in any given sample were normalized to the GAPDH signal for that sample.

Western blots analysis. Cells wee harvested in ice-cold RIPA solubilization buffer, and total amounts of protein were determined using a bicinchoninic acid quantification assay (Pierce, Rockford, Ill.). Fifty μg of total protein/lane were separated by SDS/PAGE under reducing conditions and blotted to polyvinylidene difluoride membranes (Millipore Corp., Bedford, Mass.) using a semidry blotting apparatus (BioRad, Hercules, Calif.). Blots were blocked overnight in 5% defatted dry milk/PBS/0.1% Tween, and then incubated for 1 hour at room temperature with primary antibody (1:200) (mouse monoclonal anti-human p-Stat1α Santa Cruz, San Diego, Calif.), or mouse monoclonal anti-human β-actin (1:5000) (Pharmingen) for control of loading. This was followed by a 1 hour incubation with secondary peroxidase conjugated antibody (1:10000), (Jackson Immunoresearch, West Grove, Pa.). All steps were performed at room temperature and in between incubation steps cells were rinsed with PBS/0.1% Tween. Immunoreactivity was detected using the enhanced chemiluminescence detection method according to the manufacturer's instructions. (Amersham, Dübendorf, Switzerland), and subsequent exposure of the membranes to x-ray film.

Cytokine assay, Release of T12 from T lymphocytes was measured using ELISA kits, as suggested by the manufacturer (R&D, Abington, UK). Experiments were performed in the presence of polymyxin B (1 μg/mL). Antibody binding was detected by adding p-nitrophenyl phosphate (1,39 mg/mL), and absorbance was measured at 405 mn in a Dynatech plate reader. The amount of IL2 detected was calculated from a standard curve prepared with human recombinant IL-2. Samples were assayed in triplicate.

### Results

As part of an exploration of possible interfaces between immune mechanisms and parthenogenesis, and to evaluate possible beneficial effects of statins independently of their well-known effect as lipid lowering agents, the effect of statins on various features of the control of MHC class II expression and of subsequent lymphocyte activation has been analyzed.

The effect of several was studied on the regulation of both constitutive MTC class II expression in highly specialized antigen presenting cells (APC) and inducible MHC class II expression by interferon gamma (IFN-γ) in a variety of other cell types, including primary cultures of human endothelial cells (ECs) and monocyte-macrophages (Mφ).

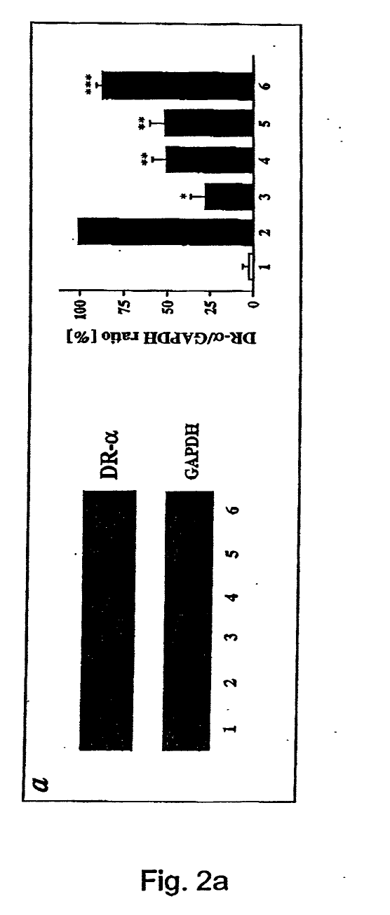

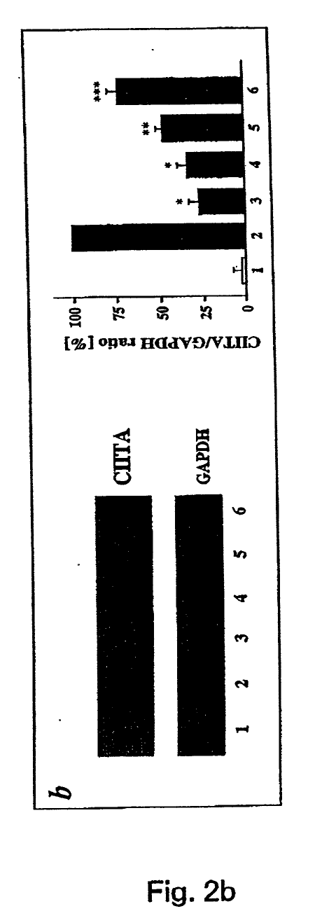

Experiments were performed to monitor cell surface expression (assayed both by FACS, FIGS. 1a-f, and by immunofluorescence, FIG. 1g, as well as mRNA levels (RNAse protection assay, FIG. 2a) of MHC class II. These investigations have led to the following conclusions: 1) Statins effectively repress the induction of MHC-II expression by IFN-γ and do so in a dose-dependant manner (FIGS. 1a-b, g). 2) In the presence of L-mevalonate, the effect of statins on MHC class II expression is abolished, indicating that it is indeed the effect of statins as HMG-CoA reductase inhibitors that mediates repression of MHC class II (FIG. 1c). 3) Interestingly, repression of MHC class II expression by statins is highly specific for the inducible form of MHC-II expression and does not concern constitutive expression of MHC-II in highly specialized APCs, such as dendritic cells and B lymphocytes (FIGS. 1d, e). 4) This effect of statins is specific for MHC class II and does not concern MHC class II expression (FIG. 1f). 5) In order to investigate functional implications of statin-induced inhibition of MHC class II expression, we performed mix lymphocyte reactions (allogenic T lymphocytes incubated with IFN-γ-pretreated human ECs or Mφ). T cell proliferation could be blocked by anti-MHC class II mAb (monoclonal antibody). Pretreatment of ECs or Mφ with statins represses induction of MHC class II and reduces subsequent T lymphocyte activation and proliferation measured by thymidine incorporation (FIG. 3a) or IL-2 release (FIG. 3b).

The novel effect of statins as MHC class II repressor was also observed and confirmed in other cell types, including primary human smooth muscle cells and fabroblasts, as well as in established cell lines such as ThP1, melanomas and Hela cells. This effect of statins on MHC class II induction is observed with different forms of statins currently used in clinical medicine. Interestingly however, different statins exhibit quite different potency as MHC class II <<repressors>> (see FIG. 1a). Of the forms tested, the most powerful MHC class II repressor is Atorvastatin. The newly described effect on MHC class II repression can be optimized by screening other members of the statin family, as well as analogues of statins.

Repression of induction of MHC class II by IFN-γ, in statin treated samples, is paralleled by a reduced induction of CIITA mRNA by IFN-γ (FIGS. 2a, b), which points to an inhibition of induction of the CITTA gene by statins. Interestingly, the different degree of repression of CIITA mRNA induction observed with the different forms of statins (FIG. 2b) are reflected in the different levels of repression of MHC class II expression observed with the same drugs (FIG. 1a). This confirms the quantitative nature of the control of CIITA over MHC class II gene activity¹³. Constitutive expression of MHC class II, known to be mediated by CIITA promoters I and III, is not affected by statins (FIGS. 1d,e), suggesting that promoter IV may be their sites of action. Indeed, we also show that induction of expression of the first exon specifically controlled by CIITA promoter IV is affected by statins (FIG. 4a). Finally, the statin effect is transcriptional, as demonstrated by actinomycin D experiments used to block de novo RNA synthesis and explore mRNA half-life (FIG. 4b), and it is direct and does not require de novo protein synthesis, as seen by a lack of effect of cycloheximide experiments.

As expected from the lack of statin effect on MHC class I induction (which is known to require Stat1α)¹⁴ the statin effect reported here is not due to an impairment of Stat1α activation, as phosphorylation and nuclear translocation of Stat1α occurs normally under the effect of statins (FIG. 4c).

### Example 2

### Statins Reduce CD40 Expression

### Materials and Methods

Reagents. Human recombinant IFNγ was obtained from Endogen (Cambridge). The statins used in those studies Atorvastatin, [Parke Davis]; Simvastatin and Lovastatin [Merck Sharp and Dohme]; and Pravastatin [Bristol Meyers Squibb]) are commercially available and were obtained from commercial sources. Because endothelial cells lack lactonases to process simvastatin, atorvastatin and lovastatin to their active forms, these agents were chemically activated before their use as previously described[Blum, 1994, 53] Rabbit anti-human CD40 polyclonal Ab, fluorescein isothiocyanate-conjugated (FITC) anti-rabbit Ab, and HRP goat anti-rabbit Ab were purchased from Santa Cruz. (Santa Cruz) Jackson ImmunoResearch (West Grovel) and Vector (Burlingame), respectively. FITC-conjugated hamster anti-mouse CD40 monoclonal antibody and FITC-conjugated hamster anti-mouse IgM were purchased by Pharmingen (San Diego). L-mevalonate was purchased from Sigma (St. Louis). Human recombinant CD40 ligand (rCD40L) was a gift from Dr. P. Graber (Serono Pharmaceutical, Geneva, Switzerland) and generated as described previously[Mazzei, 1995, 54]. Antibodies for IL-6, IL-8 and MCP-1 were obtained from R&D (Oxon).

Cell isolation and culture. Human vascular endothelial cells (ECs) were isolated from saphenous veins and mammary arteries by collagenase treatment (Worthington Biochemicals), and cultured in dishes coated with gelatin (Difco) as described elsewhere [15]. Cells were maintained in medium 199 (M199; BioWhittaker) supplemented with 100 U/ml penicillin/streptomycin (BioWhittaker), 5% FCS (Gibco), 100 μg/ml heparin (Sigma) and 50 μg/ml ECGF (endothelial cell growth factor, Pel-Freez Biological). Human vascular smooth muscle (SMCs) cells were isolated from human saphenous veins and mammary arteries by explant outgrowth, and cultured in DMEM (BioWhittaker) supplemented with 1% L-glutamine (BioWhittaker), 1% penicillin/streptomycin, and 10% FCS. Both cell types were subculture following trypsinization (0.5% trypsin (Worthington Biochemicals)/0.2% EDTA (EM Science)) in P100-culture dishes (Becton Dickinson). Culture media and FCS contained less than 40 pg LPS/ml as determined by chromogenic Limulus amoebocyte-assay analysis (QLC-1000; BioWhittaker). ECs and SMCs were >99% CD31 and α-actin (Dako) positive, respectively, as characterized by flow cytometry and were used at passages two to four for all experiments.

The human Raji cell line (Epstein-Barr virus-positive Burkitt lymphoma cell line) obtained from American Type Culture Collection (Rockville) were grown in RPMI-1640 medium containing 10% FCS.

Human monocytes were isolated from freshly prepared human peripheral blood mononuclear cells obtained from leukopacs of healthy donors following Ficoll-Hypaque gradient and subsequent adherence to plastic culture flasks (90 min., 37° C.). Monocytes were cultured in RPMI 1640 medium (BioWhittaker) containing 10% FCS of 10 days[Kwak, 2001, 31]. Macrophages (MΦ)) derived from monocytes were >98% CD64 positive as determined by flow cytometry.

Mouse monocytes were obtained by peritoneal lavage as described. Animals were on high cholesterol diet (1.25%) for then days before harvesting[Kol, 1998, 55]. Cells were grown in RPMI 1640 medium (BioWhittaker) containing 10% FCS for 10 days.

Western blots analysis. Cells were harvested in ice-cold RIPA solubilization buffer, and total amounts of protein were determined using a bicinchoninic acid quantification assay (Pierce, Rockford, Ill.). Twenty μg of total protein/lane were separated by SDS/PAGE under reducing conditions and blotted to polyvinylidene difluoride membranes (Millipore Corp., Bedford, Mass.) using a semidry blotting apparatus (BioRad, Hercules, Calif.). Blots were blocked overnight in 5% defatted dry milk/PBS/0.1% Tween, and then incubated for 1 hour at room temperature with primary antibody (1:40) (rabbit polyclonal anti-CD40 Santa Cruz, San Diego, Calif.), or mouse monoclonal anti-human β-actin (1:5000) (Pharmingen) for control of loading. This was followed by a 1 hour incubation with secondary peroxidase-conjugated antibody (1:10'000), (Jackson Immunoresearch, West Grove, Pa.). All steps were performed at room temperature and in between incubation steps cells were rinsed with PBS/0.1% Tween. Immunoreactivity was detected using the enhanced chemiluminescence detection method according to the manufacturer's instructions. (Amersham, Dübendorf, Switzerland), and subsequent exposure of the membranes to x-ray film. Analysis of quantification of detection was performed using AIDA software.

Cytokines assay. Release of IL-6, IL-8 and MCP-1 from experiments, was measured using a sandwich-type ELISA as suggested by the manufacturer (R&D system, Abingdon, UK). Experiments were performed in the presence of polymyxin B (1 μg/ml). Antibody binding was detected by adding substrate (R&D), and absorbance measured at 450 nm using a Dynatech plate reader. The amount of IL-6, IL-8 and MCP-1 detected was calculated from a standard curve prepared with the recombinant protein. Samples were assayed in duplicates.

Immunolabelins. Human and mice macrophages grown on coverslips, were rinsed and fixed for 15 min with paraformaldehyde (4%) at room temperature (RT). Coverslips were rinsed and cells incubated successively in 0.5 M NH₄Cl/PBS for 15 min and PBS supplemented with 2% bovine serum albumin (Sigma) for another 20 min. Human macrophages were then incubated overnight with primary antibody (1:50) in 10% normal goat serum (Sigma)/PBS). Mice macrophages were incubated during 2 hrs with the primary antibody FITC. After rinsing, human macrophages were incubated with secondary antibodies FITC-conjugated (1:800) for 3 hrs. All steps were performed at room temperature and between incubation steps cells were rinsed with PBS. Cells were counterstained with 0.03% Evans blue/PBS. Finally, coverslips were mounted on slides in Vectashield (Vector Laboratories, Burlingame, Calif.). Cells were examined using a Zeiss Axiophot microscope equipped with appropriate filters. Replacement of the primary antibody with PBS/10% normal goat serum or IgM-FITC were used to control the specificity of the immunolabeling of the human macrophages and mice macrophages respectively.

Human immunochemistery Surgical specimens of human carotid atheroma were obtained by protocols approved by the Investigation Review Committee at the University Hospital Geneva from patients treated or not with the statin Atorvastatin. Serial crystal sections (5 μm) were cut, air dried onto microscope slides (Fisher Scientific), and fixed in acetone at −20° C. for 5 min. Sections were preincubated with blocking buffer (PBS/Tween with 8% of normal horse serum) and then incubated successively with CD40 Ab (goat anti-human) (Santa Cruz) for 1 hour. Finally sections were incubated with biotinylated secondary Ab (45 min; Vector Laboratories) followed by with avidine-biotin-alcaline phosphatase complex (vectastain ABC kit). And antibody binding was visualized with alkaline phosphatase substrate (Vector Laboratories). Cells were not counterstained. Replacing the primary antibody with blocking buffer checked for specificity of the immnunolabeling. Analysis of immunochemistery for CD40 was performed with a computer-based quantitative color image analysis system. A color threshold mask for immunostaining was defined to detect the red color by sampling, and all the same threshold was applied to all specimens.

Flow cytometry. Cells were incubated with FITC-conjugated specific antibody (60 min, 4° C.) and analyzed in a Becton Dickinson FACScan flow cytometer as described¹⁵. At least 20,000 viable cells were analyzed per condition. Data were analyzed using CELLQUEST software (Becton Dickinson).

### Results

In order to study the effect of statins on IFN-γ induced CD40 expression, confluent vascular endothelial cells (Ecs) were cultured in the presence of 500U/ml IFN-γ in combination with simvastatin, lovastatin, pravastatin and atorvastatin. Surface CD40 expression was analysed by western blotting after 24 hrs. As can be observed in FIG. 6, ECs did express CD40 under resting conditions and IFN-γ treatment induced expression of this molecule. But with co-treatment by IFN-γ and statins, CD40 expression is decreased. Same results were obtained by FACS analysis. Interestingly statins did not shown any effects by FACS analysis on B lymphocytes (Raji) that constituvely express CD40.

Atorvastatin repressed this induction of CD40 in a dose-dependant maker (FIG. 7). The effect of Atorvastatin was observed over a range of 0.08-5 μM. Treatment with Atorvastatin alone had an effect on CD40 expression. HMG-CoA reductase inhibitors, such as Atorvastatin, block the rate-limiting enzyme in the cholesterol synthesis pathway, preventing the production of L-mevalonate. In the presence of L-mevalonate, the effect of Atorvastatin on IFN-γ induced CD40 was markedly reduced.

To investigate the functional consequences of inhibition of CD40 expression by statins on Endothelial Cells activation by CD40L, secreted cytokines were analysed such as Interleukin-6 (IL-6), interleukin-8 (IL-8), macrophages chemoattractant protein-1 (MCP-1). Addition of an anti-CD40LmAb blocked the induction of all three secreted cytokines in response to CD40 ligation.

Cytokines were measured by ELISA after 24 hrs. As can be observed in FIGS. 8a, b, c, cytokines are secreted under resting conditions, addition of simvastatin largely reduces the secretion. CD154 treatment induced expression of this molecule. But by CD154 stimulation with statins, CD40 expression is significantly decreased. Addition of L-mevalonate significantly reverses the process.

To determine whereas statins did affect macrophages, an immunofluorescence was performed. The control condition showed a basic level of CD40 which was induced by stimulation with IFN-γ. As expected addition of statins reduced the expression induced by IFN-γ and addition of L-mevalonate.

Arteries carotids plaques were analysed by immunostaining. Patients under statins treatment present less inflammatory plaques ad present less CD40 expression.

### Discussion

Increasing evidence supports the central role of CD40L-CD40 signaling pathway responses in several immuno-inflammatory processes, including atherosclerosis, graft-versus-host disease, multiple sclerosis, as well as autoimmune diseases like lupus nephritis, spontaleous autoimmiune diabetes, collage-induced arthritis.

Reducing IFN-γ induced CD40 expression with statins decreases release of chemokines (MCP-1), cytokines (IL-6, IL-8). Thus might also decrease proagulant activity (tissue factor) (that leads to the thrombus formation), MMPs (that are able to digest the compounds of the matrix and thus participate at the fibrous cap weakening), adhesion molecules as well as B cell activation that could explain plaque stabilization.

In this preset invention it is shown that statins decreased IFN-γ induced CD40 expression on vascular cells and thus reduce inflammation induced by the ligation with its ligand.

### Example 3

### Influence of Statin (Atorvastatin) on Mouse Skin Grafts

Mouse skin graft are harvested from the back region (˜2 cm²) of the animal and transplanted in the same back area of the recipient mice, stitched with 4.0 Ethibond (Johnson & Johnson). The procedures are performed in ˜20 min, under gaz anesthesia (Halothan) to avoid any suffering of the animals. Once they recovered, the animals are replaced in their cage (one animal per cage).

Control of the skin graft transplantation procedure were performed on mouse from the same strain, even the same nest (brothers and sisters). Skin transplantation was also performed on the same mouse (being the donor and the recipient) for internal controls of the transplantation procedure.

Then, skin graft transplantation was performed in mouse from two different strains (black mice from the strain C57/B16 to white mice from the strain BALB/C, and vice versa).

Soon after the transplantation, the mice were randomised and divided in three different treatment groups:

1) control

2) Low statin dose (see below for the way of administration)

3) High statin dose (see below for the way of administration)

7 mice per group were performed.

Skin graft transplantation was analysed at day 7, 10 and 14 after the procedure (FIG. 10).

At day 7, 10 and 14 after transplantation, rejection was defined and measured in all mice (granulation tissue and vascularisation) at the site where the graft were placed, using a Laser Doppler Perfusion Image (LDPI) system (Lisca, Inc).

At day 20, all the mice were sacrificed, the skin graft piece including recipient tissue isolated and embedded and frozen in OCT for immunohistochemical analysis.

Internal controls:

Mice did not change weight significantly between groups.

Blood cholesterol levels (total cholesterol, triglycerides) did not change during the experiments from control group compared to low statin dose. Mice in the high statin treatment group showed a slight decrease for these blood measurements.

Statin treatment (in melted food):

| | |
|---|---|
| Atorvastatin human dose: | 80 mg/day for ≈ 80 kg (1 mg/kg) |
| Mouse weight: | 20 gr |
| Mouse food: | ≈10 g/day |
| Dose 1 (low): | 1 mg/kg/day 20 μg/day/mice |
| Dose 2 (high): | 100 mg/kg/day 2 mg/day/mice |

Atorvastatin stock solution:

1) 200 mg in 20 ml H₂O i.e. 10 mg/ml

2) 2 mg in 20 ml H₂O i.e. 100 μg/ml

Food preparation 1: 110 gr of food+115 ml H₂O+3 ml (30 mg) of stock solution 1

Food preparation 2: 110 gr of food+115 ml H₂O+3 ml (30 mg) of stock solution 2

Dose 1: For a cage of 5 mice: 75 gr/per day of the food preparation 1 (above)

Dose 2: For a cage of 5 mice: 75 gr/per day of the food preparation 2 (above)

### Example 4

### Statins in the Treatment of Inflammatory Diseases

i) Effect of Statins in Mice with Collagen-Induced Arthritis

Collagen-induced arthritis is a well-described animal model that produces some of the typical clinical and pathological features of human RA (32). DBA/1 mice are typically used in this model and develop arthritis within four to eight weeks after immunization. Histological findings in CIA include the presence of inflammatory cells in the synovial membrane and synovial fibroblast proliferation with pannus formation and subsequent cartilage and bone destruction, mimicking the pathological features of RA. This experimental model of arthritis is available in the laboratory of the Division of Rheumatology (University of Geneva). The effect of the administration of statins in the frequency and severity of CIA development can thus be examined. DBA/1 mice are used for this experiment. For the treated group, statins are added in the drinking water. Atorvastatin is used at 1 mg/kg/day and 20 mg/kg/day compared to controls (untreated nice). These doses of statin treatment are usual for mice models, such as for the atherosclerosis one currently in investigation in laboratory of Dr. F. Mach (Division of Cardiology, University of Geneva). The mice are then injected with bovine collagen type II in complete Freund's adjuvant with a subsequent booster injection after 21 days as recently described (33). The animals are examined 3 times per week for the appearance and severity of arthritis using the index described (33). The results within each group (incidence of arthritis, joint swelling, and extent of joint disease) are used for statistical analysis. The model of collagen-induced arthritis are performed by the laboratory of Dr. C. Gabay (Division of Rheumatology, University of Geneva). At the termination of the study (eight weeks after the first injection), the mice are sacrificed and their paws removed for histological examination. The limbs are removed, fixed, decalcified, and stained with hematoxylin and eosin. The histological alterations, particularly the presence of pannus and signs of cartilage degradation are examined. The results obtained in each group are compared. Histology and immunohistology staining for expressing of MHC.II, inflammatory cell subtypes, and cytokines are performed. The experiments are repeated two times for accurate statistical analysis. In addition, some mice are sacrificed during the course of the study at different stages of the disease. Total RNA from the joint are prepared and mRNA levels for different cytokines and chemokines arc determined by RNase protection assay. In addition, as a marker of the inflammatory response, plasma levels of serum amyloid A, a major acute phase protein in the mouse are measured by ELISA. Preliminary results obtained in mice with CIA indicated that circulating levels of serum amyloid A correlate with the presence of arthritis. All these results are used to compare the local and systemic inflammatory responses between treated and control mice.

The effect of atorvastatin on the cellular and humoral components of the immune response is be examined. Inguinal lymph nodes from treated and control mice arc removed 14 days after the immunization. Lymph node T-cells are prepared and stimulated in vitro with bovine collagen type II. T-cell proliferation are assessed by 3H-thymidie uptake. In addition, the production of interferon-γ by stimulated T lymphocytes is measured by ELISA in the cell supernatants. The effect of atorvastatin on the humoral response is studied by measuring the levels of circulating anti-bovine collagen type II antibodies.

The effect of statin on the curse of CIA is also examined by introducing the treatment with atorvastatin in mice at the onset of arthritis. For this purpose, atorvastatin is added at the moment of the booster injection of bovine collagen type II. Indeed, the occurrence of overt arthritis is detected in days after this booster injection. The same parameters are used as those described above to define the severity of arthritis, the immune-mediated response, as well as the signs of joint damage.

Protocol:

Study A: 3 groups of 10 mice (3×2 cages of 5 mice) separated in control, low and high statin dose. Rheumatoid arthritis joint deformation is evaluated after 2^{nd} immunization.

Study B: 3 groups of 5 mice (3×1 cage of 5 nice) separated in control, low and high statin dose. Soon after 2^{nd} immunization, inguinal lympho-nodes is isolated and analysed (T lymphocyte proliferation, IFN-γ production).

All mice arc separated and randomised (control, low and high statin groups) at arrival.

For all mice, first immunization is performed the same day, and second immunization 21 days later.

Statin treatment (in melted food):

| | |
|---|---|
| Atorvastatin human dose: | 80 mg/day for ˜80 kg (1 mg/kg) |
| Mouse weight: | 20 gr |
| Mouse food: | ˜10 g/day |
| Dose 1 (low): | 1 mg/kg/day 20 μg/day/mice |
| Dose 2 (high): | 100 mg/kg/day 2 mg/day/mice |

Atorvastatin stock solution:

1) 200 mg in 20 ml H₂O i.e. 10 mg/ml

2) 2 mg in 20 ml H₂O i.e. 100 μg/ml

Food preparation 1: ∠gr of food+115 ml H₂O+3 ml (30 mg) of stock solution 1

Food preparation 2: ∠gr of food+115 ml H₂O +3 ml (30 mg) of stock solution 2

Dose 1: For a cage of 5 mice: 75 gp/per day of the food preparation 1 (above)

Dose 2: For a cage of 5 mice: 75 gr/per day of the food preparation 2 (above)

ii) Effect of Statins in a Pilot 12-Week Open Clinical Trial in Patients with RA.

Rheumatoid arthritis is a severe inflammatory disease that is characterized by a poor or incomplete response to classical treatments leading to joint destruction and invalidity in 80% of the cases after 20 years of evolution. Since the last decade, it has become extremely clear that aggressive treatment such as the combination of two or three different disease modifying anti-rheumic drugs (DMARDs) is required to control the disease activity in several patients. The step-up approach, the addition of a second or a third DMARDs, is generally used by most rheumatologists. The aim of this study is thus to show that statins provide an additional effect to classical DMARDs. For this purpose, patients with RA that have clinical signs of active disease despite treatment with DMMRs are included in this study. Active RA is defined by the presence of 4 or more swollen joints, 4 or more tender joints and at least one of the following morning stiffness that last 45 minutes and a serum CRP concentration of at least 20 mg per liter.

For this pilot study, 20 RA patients fulfilling the 1987 ACR criteria for RA and the eligibility criteria defined above are enrolled. The presence of severe extra-articular manifestations such as rheumatoid vasculitis requiring an immunosuppressive treatment is considered as an exclusion criteria. Determination of lipid levels is determined at the study entrance (cholesterol, HDL-c, LDL-c, triglyceride). Patients continue to receive the same DMARDs treatment as before the study and also receive 80 mg Atorvastatin/day. This dosage has already been used in three recent clinical trials (3436). In addition, it has been shown that the effect of Atorvastatin on biological markers of inflammation dosce-dependent and that a decrease in CRP levels were observed at a dosage of 80 mg/day. Patients are allowed to continue the same dose of DMARDs, non-steroidal anti-inflammatory drug and oral glucocorticoids (prednisone ≦10 mg/day) they bad been using before the study entry. Each patient included in the study sign an informed consent. The protocol of this study is submitted to the ethical committee of the University Hospital of Geneva.

The clinical evolution at 12 and 24 weeks is assessed by the same investigator. A clinical response is defined according to the ACR definition of a 20 percent (50 percent and 70 percent) improvement. The ACR criteria of improvement included the number of tender and swollen joints, the patient's global assessment of status, the patient's assessment of pain and the physician's global assessment of disease status, all of which are assessed with the use of visual-analogue scales (VAS). Arthritis functional disability will be measured with the Health Assessment Questionnaire (HAQ), a well-defined, self-administered form. The response is also assessed by the ESR and the serum concentrations of CRP.

In addition, blood is collected at the inclusion (before the start of the treatment) and at the end of the study (12 weeks). Serum is prepared and stored frozen (−80° C.) until used for cytokine and chemokine determinations.

Side effects related to statins include the occurrence of myalgias with elevation of creatine kinase and of hepatitis. As previously used in most studies, the serum levels of Creatine Kinase (CK) and transaminases (ASAT/ALAT) are examined at the start of the study and during the course of the treatment after 12 and 24 weeks. Treatment is stopped if transaminases are ≦3× the upper limit and if CK are ≦10× above upper limit. It is important to mention that determination of serum levels of liver transaminases arc included in the follow-up of RA patients treated with methotrexate or sulphasalazine, the two most commonly DMARDs.

iii) Effect of Statins in a Pilot 24-Week Open Clinical Trial in Patients with RA.

According to the results obtained with this preliminary study, a 24 week randomized double-blind clinical trial with atorvastatin is carried out. For this study, patients are randomly assigned to receive the same DMARDs treatment before the study plus statin or a placebo. Patients are allowed to continue the same dose of non-steroidal anti-inflammatory drug and oral glucocorticoids (prednisone ≦10 mg/day) they had been using before the study entry. Each patient included in the study signs an informed consent. The protocol of this study is submitted to the ethical committees concerned.

Exclusion criteria: Serum cholesterol concentration is measured in patients eligible for this study. Patients with a positive history of coronary arterial disease and a serum level of total cholesterol ≦7 mmol/L are excluded. The presence of severe extra-articular manifestations such as rheumatoid vasculitis requiring an immunosuppressive treatment are also considered as an exclusion criteria.

The clinical evolution at 12 and 24 weeks is assessed by independent assessors who have no knowledge of patient's treatment by using the parameters described above. In addition, the levels of cytokines and chemokines arc examined and correlation with clinical parameters are performed.

### REFERENCES

1. Maron, D J., Fazio, S. & Linton, M. F. Current perspectives on statins. Circulation 101, 207-213 (2000).

2. Yaughan, C. J., Gotto, A. M. & Basson, C. T. The evolving role of statins in the management of atherosclerosis. J. Am. Coll. Cardol. 35, 1-10 (2000).

3. Pedersen, T. R. Statin trials and goals of cholesterol-lowering therapy after AMI. Am. Heart. J. 138, 177-182 (1999).

4. Kobashigawa, J. A. et al. Effect of pravastatin on outcomes after cardiac transplantation. N. Engl. J. Med. 333, 621-627 (1995).

5. Mach, B., Steimle, V., Martinez-Soria, E. & Reith, W. Regulation of MHC class II genes: lessons from a disease. Annu. Rev. Immunol. 14, 301-331 (1996).

6. Steimle, V. et al. Complementation cloning of an MHC class II transactivator mutated in hereditary MHC class II deficiency (or bare lymphocyte syndrome) Cell 75, 135-146 (1993).

7. Steimle, V. et al. Regulation of MHC class II expression by interferon-gamma mediated by the transactivator gene CIITA. Science 265, 106-109 (1994).

8. Hebert, P. R., Gaziano, J. M., Chan, K. S. & Hennekens, C. H. Cholesterol lowering with statin drugs, risk of stroke, and total mortality. An overview of randomized trials. JAMA 278, 313-21 (1997).

9 Kwak B, Mulhaupt F, Veillard N, Pelli G, Mach F. HMG-CoA reductase inhibitor sinvastatin inhibits IFN-g induced MHC class II expression in human vascular endothelial cells. Swiss Medical Wkly 131; 41-46 (2001).

10. Mastemak, K. et al. A gene encoding a novel RFX-associated transactivator is mutated in the majority of MHC class II deficiency patients. Nat. Genet. 20, 273-277 (1998).

11. Muhbethaler-Mottet, A. et al. Expression of MHC Class II molecules in different cellular and functional compartments is controlled by differential usage of multiple promoters of transactivator CIITA. EMBO J. 16, 2851-2860(1997).

12. Muhlethaler-Mottet, A. et al. Activation of MHC Class II transactivator CIITA by interferon gamma requires cooperative interaction between Stat1 and USF-1. Immunity 8, 157-166 (1998).

13. Otten, L. A, Sterile, V., Bontron, S. & Mach, B. Quantitation control of MHC Class II expression by the transactivator CIITA. Eur. J. Immunol. 2, 473-478 (1998).

14. Lee, Y. J., & Benveniste, E. N. Stat1 alpha expression is involved in IFN-gamma induction of the class II transactivator and class II MHC genes. J. Immunol. 157, 1559-1568 (1996).

15. Mach, F. et al. Functional CD40 is expressed on human vascular endothelial cells, smooth muscle cells, and macrophage: Implication for CD40-CD40 ligand signaling in atherosclerosis. Proc. Natl. Acad. Sci. US. 94, 1931-1936 (19.97).

16. Arrighi, J. F., Hauser, C., Chapuis, B., Zubler, R. H. & Kindler, V. Long-term culture of human CD34(+) progenitors with FLT3-ligand, thrombopoictin, and stem cell factor induces extensive amplification of a CD34(−)CD14(−) and a CD34(−)CD14(+) dendritic cell precursor. Blood 93, 2244-2252 (1999).

17. McPherson, R., Tsoukas, C., Baines, M. G., Vost, A., Melino, M. R., Zupkis, R. V. & Pross, H. F. Effects of lovastin on natural killer cell function and other immunological parameters in man, J. Clin. Immunol. 13, 439-444 (1993).

18. Cutts, J. L. & Bankhurst, A. D. Suppression of lymphoid cell function in vitro by inhibition of 3-hydroxy-3-methylglutaryl coenzyme A reductase by lovastatin. Int. J. Immunopharmacol. 11, 863-869 (1989).

19. Roth, B. D., Bocan, T. M. A., Blankley, C. J., et al. Relation between tissue selectivity and lipophilicity for inhibitors of HMG-CoA reductase. J. Med. Chem. 34, 463-466 (1991).

20. Shaw, M. K., Newton, R. S., Sliskovic, D. R, Roth, B. D., Ferguson, E. & Krause, B. R. Hep-G2 cells and primary rat hepatocytes differ in their response to inhibitors of HMG-CoA reductase. Biochem. Biophys. Res. Commun. 170, 726-734 (1990).

21. Arend, W. P., Dayer, J -M., Inhibition of the production and effects of interleukin-1 and tumor necrosis factor □ in rheumatoid arthritis. Arthritis Rheum. 38, 151-160 (1995).

22. Jiang, Y., Genant, H. K., Watt, I., Cobby, M., Bresnihan, B., Aitchison, R., et al, A multicenter, double-blind, dose-randing, randomized, placebo-controlled study of recombinant human interleukin-1 receptor antagonist in patients with rheumatoid arthrisis. Arthritis Rheum. 43, 1001-1009 (2000).

23. Lispky, P. B., et al, Infliximab and methotrexate in the treatment of rheumatoid arthisis. N. Engl J. Med., 343, 1594-1602 (2000).

24. Libby, P. Changing concepts of atherogenesis. J. Intern Med 247, 349-58. (2000).

25. Kwak, B., Mullhaupt, F., Myit, S., Mach, F., Statins as a newly recognized type of immunomodulator, Nature Med., 6, 1399-1403 (2000).

26. Lusis, A. J. Atherosclerosis, Nature 407, 233-41. (2000).

27. Glass, C. K. & Witztum, J. L. Atherosclerosis, the road ahead, Cell 104, 503-16. (2001).

28. Mach, F., Schonbeck, U. & Libby, P. CD40 signaling in vascular cells: a key role in atherosclerosis? Atherosclerosis 137 Suppl, S89-95. (1998).

29. Schonbeck, U. & Libby, P. The CD40/CD154 receptor/ligand dyad. Cell Mol Life Sci 58, 4-43. (2001).

30. Mach, F., Schoneck, U., Sukhova, G. K., Alkinson, E. & Libby, P. Reduction of athrosclerosis in mice by inhibition of CD40 signalling, Nature 394, 200-3. (1998).

31. Kwak, B., Mach, F., Statins inhibit leucocyte recruitment: new evidence for their anti-inflammatory effects. Arterioscler. Thromb. Vasc. Biol., In press (2001).

32. Stuart J. M., Townes A. S., Kang, A. H., Collagen autoimmune arthrisis. Annu. Rev. Immunol., 2, 190-199 (1984).

33. Gabay, C., Marinova-Mutafchieva, L., Williams, R. O., Gigley, J. P., Butler, D. M., Feldmann, M., et al, Increased production of intracellular interleukin-1 receptor antagonist type I in the synovium of mice with collagen-induced arthritis. A possible role in the resolution of arthritis. Arthritis Rheum., 44, 251-262 (2001).

34. Pitt, B., Waters D., Brown, W. V., Boven, A. J., Schwarz, L., Title, L. M., et al, The Atorvastatin versus revascularization treatment investigators, N. Engl. J. Med., 341, 70-76 (1999).

35. Smilde T. J., Van Wissen S., Trip, M. D., Kastelein, J. P., Stalenhoef, A. F. H., Effect of aggressive versus conventional lipid lowering on artherosclerosis progression in familial hypercholesterolemia: a prospective, randomized, double-blind trial. Lancet, 357, 577-581 (2001).

36. Schwarz C., Olsson A. G, Ezekowitz M. D, Ganz, P., Olivier, M. F., Waters, D., et al, The myocardial ischemia reduction with aggressive cholecterol lowering (MIRACL) study investigators, JAMA, 235, 1711-1718 (2001).

Lusis, A. J. Atherosclerosis. Nature 407, 233-41. (2000).

37. Lutgens, E. et al. Requirement for CD154 in the progression of atherosclerosis. Nat Med 5, 1313-6. (1999).

38. Schonbeck, U., Sukhova, G. K, Shimizu, K, Mach, F. & Libby, P. Inhibition of CD40 signaling limits evolution of established atherosclerosis in mice. Proc Natl Acad Sci US A 97, 7458-63. (2000).

39. Durie, F. H. et at. Prevention of collagen-induced arthritis with an antibody to gp39, the ligand for CD40. Science 261, 1328-30. (1993).

40. Gerritse, K. et al. CD40-CD40 ligand interactions in experimental allergic encephalomyelitis and multiple sclerosis. Proc Natl Acad Sci USA 93, 2499-504. (1996).

41. Jensen, J., Krakauer, M. & Sellebjerg, F. Increased T cell expression of CD154 (CD40-ligand) in multiple sclerosis. Eur J Neurol 8, 321-8. (2001).

42. Shimizu, K., Schonbeck, U., Mach, F., Libby, P. & Mitchell, R. N. Host CD40 ligand deficiency induces long-term allograft survival and donor-specific tolerance in mouse cardiac transplantation but does not prevent graft arteriosclerosis. J Immunol 165, 3506-18. (2000).

43. Larsen, C. P. et al. Long-term acceptance of skin and cardiac allografts after blocking CD40 and CD28 pathways. Nature 381, 434-8. (1996).

44, Karmann, K., Hughes, C. C., Schechner, J., Fanslow, W. C. & Pober, J. S. CD40 on human endothelial cells: inducibility by cytokines and functional regulation of adhesion molecule expression. Proc Natl Acad Sci USA 92, 4342-6. (1995).

45. Hollenbaugh, D. et al. Expression of functional CD40 by vascular endothelial cells. J Exp Med 182, 33-40. (1995).

46. Yellin, M. J. et al. Functional interactions of T cells with endothelial cells: the role of CD40L-CD40-mediated signals. J Exp Med 182, 1857-64. (1995).

47. Mach, P., Schonbeck, U., Bonnefoy, J. Y., Pober, J. S. & Libby, P. Activation of monocyte/macrophage functions related to acute atheroma complication by ligation of CD40: induction of collagenase, stromelysin, and tissue factor. Circulation 96, 396-9. (1997).

48. Mach, F. et al. T lymphocytes induce endothelial cell matrix metallloproteinase expression by a CD40L-dependent mechanism: implications for tubule formation. Am J Pathol 154, 229-38. (1999).

49. Schonbeck, U. et al. Regulation of matrix metalloproteinase expression in human vascular smooth muscle cells by T lymphocytes: a role for CD40 signaling in plaque rupture? Circ Res 81, 448-54. (1997).

50. Schonbeck, U. et al. CD40 ligation induces tissue factor expression in human vascular smooth muscle cells. Am J Pathol 156, 7-14. (2000).

51. Mach, F. et al. Differential expression of three T lymphocyte-activating CXC chemokines by human atheroma-associated cells. J Clin Invest 104, 1041-50. (1999).

52. Sugiura, T. et al. Increased CD40 expression on muscle cells of polymyositis and dermatomyositis: role of CD40-CD40 ligand interaction in IL-6. IL-8, II-15, and monocyte chemoattractant protein-1 production. J Immunol 164, 6593-600. (2000).

53. Blum, C. B. Comparison of properties of four inhibitors of 3-hydroxy-3-methylglutaryl-coenzyme A reductase. Am J Cardiol 73, 3D-11D. (1994).

54. Mazzei, G. J. et al. Recombinant soluble trimeric CD40 ligand is biologically active. J Biol Chem 270, 7025-8. (1995).

55. Kol, A., Sukova, G. K., Lichtman, A. H. & Libby, P. Chlamydial heat shock protein 60 localizes in human atheroma and regulates macrophage tumor necrosis factor-alpha and matrix metalloproteinsse expression. Circulation 98, 300-7. (1998).

1- A method to achieve MHC-class II mediated immunomodulation in a mammal in need of such treatment, which comprises administering to the mammal at least one statin, or a functionally or structurally equivalent molecule, in an amount effective to modulate MHC class II expression in the mammal.

2- A method to achieve MHC-class II mediated immunosuppression in a mammal in need of such treatment, which comprises administering to the mammal at least one statin, or a functionally or structurally equivalent molecule, in an amount effective to suppress MHC class II expression in the mammal.

3- A method to achieve MHC-class II mediated anti-inflammatory effect in a mammal in need of such treatment, which comprises administering to the mammal at least one statin, or a functionally or structurally equivalent molecule, in an amount effective to suppress MHC class II expression in the mammal.

4- A method to achieve CD40-mediated anti immuno-inflammatory effect in a mammal in need of such treatment, which comprises administering to the mammal at least one statin, or a functionally or structurally equivalent molecule, in an amount effective to modulate CD40 expression.

5- The method of any one of claims 1 to 4, wherein said mammal is a human.

6- The method of any one of claims 1 to 4, wherein said mammal does not suffer from hypercholesterolaemia.

7- The method or claims 1, 2 or 3, wherein said amount is effective to specifically modulate IFN-γ inducible MHC class II expression.

8- The method of claim 4, wherein said amount is effective to specifically modulate inducible CD40 expression.

9- The method of claim 8, wherein said inducible CD40 expression in induced by IFN-γ.

10- The method of claims 1, 2 or 3, wherein said mammal is suffering from a condition which involves IFN-γ inducible CIITA expression.

11- The method of any one of claims 1 to 4, wherein said mammal is suffering from a condition which is an autoimmune disease.

12- The method of claim 11, wherein said autoimmune disease is type I diabetes, multiple sclerosis, rheumatoid arthritis, Crohne's disease or lupus erythematosus.

13- The method of any one of claims 1 to 4, wherein said mammal is under treatment in preparation of or after an organ or tissue transplantation.

14- The method of any one of claims 1 to 4, when said mammal is under treatment in preparation of an organ or tissue transplantation.

15- The method of any one of claims 1 to 4, wherein said mammal is suffering from a condition which is psoriasis or inflammation.

16- The method of any one of claims 1 to 4, wherein said statin is used in a topical application.

17- The method according to claim 16, wherein said topical application is on dermis or eye.

18- The method of any one of claims 1 to 4, wherein said statin is Compactin, Atorvastatin, Lovastatin, Pravastatin, Fluvasatin, Mevastatin, Cerivasin, Rosuvastati or Simvastatin.

19- The method of any one of claims 1 to 4, wherein said statin is Atorvastatin.

20- The method of any one of claims 1 to 4, wherein said statin, or said functionally or structurally equivalent molecule, has no lipid-lowering effect.

21- The method of any one of claims 1 to 4, wherein the statin or a functionally or structurally equivalent molecule, is administered in the absence of any other immunosuppressive agents.

22- The method of any one of claims 1 to 4, wherein said amount is comprised between 10 and 80 mg per day.

23- The method of any one of claims 1 to 4, wherein said amount is comprised between 20 and 40 mg per day.

24- The method of any one of claims 1 to 4, wherein said administration comprises intralesional, intraperitoneal, intramuscular or intravenous injection; infusion; or topical, nasal, oral, ocular or otic delivery.

25- The method of any one of claims 1 to 4, wherein said administration is made daily.

26- The method of any one of claims 2 to 4, wherein the immunosuppression or anti-inflammatory effect is the result of repression of T lymphocyte activation.

27- A method for identify molecules that inhibit IFN-γ induced CIITA expression, said inhibition being at least partially reversible by addition of L-mevalonate, comprising the steps of:
contacting a cell which is IFN-γ responsive with a candidate inhibitory molecule and with IFN-γ;
detecting the inhibition or absence of CIITA expression or MHC class II expression in the presence of the candidate molecule;
further contacting the cell with L-mevalonate; and
detecting a total or partial reversal of the inhibitory effect.

28- A method for identifying molecules that inhibit IFN-γ induced CIITA expression, comprising the steps of:
contacting a cell which is IFN-γ responsive with a statin, or a functional or structural equivalent thereof, and with IFN-γ;
detecting the inhibition or absence of CITTA expression or MHC class II expression in the presence of the statin, or the functional or structural equivalent thereof.

29- A method for identifying molecules that inhibit induced CD40 expression, said inhibition being at least partially reversible by addition of L-mevalonate, comprising the steps of:
contacting a cell with a candidate inhibitory molecule and with the molecule inducing CD40 expression;
detecting the inhibition of CD40 expression in the presence of the candidate molecule;
further contacting the cell with L-mevalonate; and
detecting a total or partial reversal of the inhibitory effect.

30- A method according to claim 29, wherein said molecule inducing CD40 expression is IFN-γ.

31- A method of treating a patient afflicted with an autoimmune disease, comprising administering to said patient a compound that inhibits 3-hydroxy-3-methylglutaryl coenzyme A (HMG-CoA) reductase in an amount effective to treat said disease.

32- The method of claim 32 wherein said compound has a therapeutically insignificant lipid-lowering effect and suppresses MHC Class II expression.

33- A method of treating a patient suffering from an autoimmune disease or condition comprising:
administering to said patient at least one compound, capable of measurable HMG-CoA reductase inhibition and inhibition of MHC Class II expression in said patient, in an amount effective to treat such autoimmune disease or condition.

34- A method of treating a patient in preparation for or after an organ tissue transplant comprising:
administering to said patient at least one compound capable of measurable HMO-CoA reductase inhibition and inhibition of MHC Class II expression in said patient, in an amount which is effective to prevent tissue rejection.

35- A method of preventing or treating tissue or organ rejection in a patient comprising administering to said patient a compound that inhibits 3-hydroxy-3-methylglutaryl coenzyme A reductase (HMG-CoA reductase) in an amount effective to prevent or treat tissue or organ rejection.

36- A method of treating an autoimmune disease or an immuno-inflammatory disease, comprising administration of at least one statin, or a functionally or structurally equivalent molecule, to a subject in an amount effective to modulate INF-γ inducible MHC class II expression and/or CD40 expression in the subject, such that the symptoms of said disease are at least partially alleviated.

37- A method according to claim 36, wherein the disease is rheumatoid arthritis.

38- The method of claim 36, wherein said subject does not suffer from hypercholesterolemia.

39- The method of claim 36, wherein said statin is selected from the group consisting of compactin; atorvastatin; lovatatin; pravastatin; fluvastatin; mevastatin; cerivastatin; rosuvastatin and siruvastatin; and combinations thereof.

40- The method of claim 36, wherein said statin is administered in conjunction with another rheumatoid arthritis therapy.

41- The method of claim 40, wherein said other rheumatoid arthritis therapy is selected from the group consisting of steroids; nonsteroidal anti-inflammatory agents; (NSAIDs); disease modifying anti-rheumatoid drugs (DMARDs); and combinations thereof.

42- The method of claim 41, wherein said nonsteroidal anti-inflammatory agent is selected from the group consisting of salicylates; fenoprofen; naproxen; pirxocam; tolmetin; indomethacin; sulindac; meclofenamate; and combinations thereof.

43- The method of claim 41, wherein said disease modifying anti-rheumatoid drug is selected from the group consisting of D-penicillamine; gold salts (both parenteral and oral forms); hydroxychloroquine; azathioprine; methotrexae; cyclophosphamide; and combinations thereof.

44- The method of claim 36, wherein said statin or functionally or structurally equivalent molecule, is administered orally.

45- The method of claim 36, wherein said amount is at least from about 10 and 80 mg/day.

46- The method of claim 36, wherein said amount is at least from about 20 and 40 mg/day.

47- Use of a statin or a functionally or structurally equivalent molecule, for the preparation of a medicament for treating an autoimmune disease or an immuno-inflammatory disease, such statin being present in an amount effective modulate IFN-γ inducible MHC class II expresion and/or CD40 expression, thereby alleviating at least partially the symptoms of said disease.

48- Use according to claim 47 wherein the disease is rheumatoid arthritis.

49- A method of preventing or treating tissue rejection in a subject comprising administering to said subject at least one statin or a functionally or structurally equivalent molecule in an amount which is effective to inhibit IFN-γ inducible MHC Class II expression and/or CD40 expression such that section is at least partially prevented or treated.

50- A method of treating a tissue graft prior to, during or after transplantation, comprising administering to a patient a statin or a functionally or structurally equivalent molecule, in an amount which is effective to inhibit IFN-γ inducible MHC Class II expression and/or CD40 expression effective such that inflammation or tissue rejection, or both, is reduced.

51- The method of claim 50, wherein said tissue graft is selected from the group consisting of skin; bone; abdominal wall; pericardium; periosteum; perichondrium; intervertebral disc; articular cartilage; dermis; epidermis; ligaments; bowel and tendons.

52- The method of claim 50, wherein said tissue graft is selected from the group consisting of living and synthetic graft materials.

53- The method of claim 50, wherein said statin is selected from the group consisting of compactin; atorvastatin; lovastatin; pravastatin; fluvasatin; mevastatin; cerivastn; rosuvastatin and simvastatin; and combinations thereof.

54- The method of claim 50, wherein the tissue graft is a skin graft.

55- The method of claim 54 wherein the skin graft is used for the treatment of skin ulcers.

56- The method of claim 55, wherein the skin graft is a skin allograft.

57- The method of claim 50, wherein the statin or a functionally or structurally equivalent molecule, is administered orally or topically.

58- The method of claim 50, wherein said amount is at least from about 10 and 80 mg/day.

59- The method of claim 50, wherein said amount is at least from about 20 and 40 mg/day.

60- Use of a statin or a functionally or structurally equivalent molecule in the preparation of a medicament for reducing inflammation or for reducing tissue rejection, or both, such stain being post in an amount effective to inhibit IFN-γ inducible MHC Class II expression and/or CD40 expression such that inflammation or tissue rejection, or both, is reduced, for administration to a subject before, during or after a tissue graft.

61- The use of claim 60, wherein said tissue graft is a skin graft.

62-T he use of claim 60, when said administration of said statin does not include administration of other immunosuppressive agents.

63- The use of claim 60, where said statin is selected from the group consisting of compactin; atorvastatin; lovastatin; pravastafin; flavastatin; meovastatin; cerivastatin rosuvasin and simvastatin; and combinations thereof.

64- A kit comprising a tissue graft material and a statin, or a functionally or structurally equivalent molecule, either in the same or separate packaging.

65- The kit of claim 64, wherein said tissue graft material is a natural or engineered material.

66- The kit of claim 65, wherein said tissue graft material is selected from the group consisting of skin; bone, abdominal wall; pericardium; periosteum; perichondrium; intervertebral disc; articular cartilage; dermis; epidermis; bowel; ligaments; and tendons.

67- The kit of claim 64, wherein said statin is selected from the group consisting of compactin; atorvastatin; lovastatin; pravastatin; fluvastatin; mevasiatin; cerivastatin; rosuvastatin and simvastatin; and combinations thereof.

68- A method of preventing or treating organ rejection in a subject comprising administering to said subject prior to or during transplantation, at least one statin or a functionally or structurally equivalent molecule, in an amount which is effective to inhibit IFN-γ inducible MHC Class II expression and/or CD40 expression such that rejection is at least partially prevented or treated.

69- The method of claim 68, wherein said organ is selected from the group consisting of heart, kidney, and liver.

70- The method of claim 68, wherein said statin is selected from the group consisting of compactin, atorvastatin; lovastatin; pravastatin; fluvastatin; mevastatin; cerivastatin; rosuvastatin and simvastatin, and combinations thereof.

71- The method of claim 68 wherein the organ is heart and the statin or functionally or structurally equivalent molecule, is administered to the subject prior to the transplantation.

72- The method of claim 68 wherein the organ is kidney and the statin or functionally or structurally equivalent molecule, is administered to the subject prior to the transplantation.

73- The method of claim 68, wherein the statin or functionally or structurally equivalent molecule, is administered by oral intralesional, intraperitoneal, intramuscular delivery or by intravenous injection.

74- The method of claim 68, wherein said amount is at least from about 10 and 80 mg/day.

75- The method of claim 68, wherein said amount is at least from about 20 and 40 mg/day.

76- A method of treating an inflammatory disorder comprising administering to a subject, at least one stain or a functionally or structurally valent molecule, in an amount which is effective to inhibit IFN-γ inducible MHC Class II expression and/or CD40 expression such that inflammation is reduced.

77- The method according to claim 76, wherein the inflammatory disorder is selected from the group consisting of inflammatory skin disease, inflammatory ocular disorder, and lupus erythematosus.

78- A method according to claim 76 wherein the inflammatory disorder is an inflammatory skin disorder.

79- The method of claim 76, wherein said statin is selected from the group consisting of compactin; alorvastatin; lovastatin; pravastatin; flarvastatin; mevastatin; cevastatin; rosuvastatin and simvastatin; and combinations thereof.

80- The method of claim 76 wherein the statin is administered orally or topically.

81- The method of claim 76 wherein the statin is administered topically as a gel, cream, spray, or powder.

82- The method of claim 76 wherein the inflammatory skin disease in selected from the group consisting of psoriasis and eczema.

83- The method of claim 76, wherein said amount is at least from about 10 and 80 mg/day.

84- The method of claim 76, wherein said amount is at least from about 20 and 40 mg/day.

85- Use of a statin or a functionally or structurally equivalent molecule in the preparation of a medicament for reducing inflammation in an inflammatory skin disorder, such statin being present in an amount effective for reducing inflammation.

86- A method according to claim 76 wherein the inflammatory disorder is an inflammatory ocular disorder.

87- The method of claim 86, wherein said statin is selected from the group consisting of compactin; atorvastatin; lovastatin; pravasatin; fluvastain; mevastatin; cerivastatin; rosuvastatin and simvastatin; and combinations thereof.

88- The method of claim 86 wherein the statin is administered orally or topically.

89- The method of claim 86 wherein the statin is adminstered topically as a gel, cream, spray, or powder.

90- The method of claim 86 wherein the ocular disease is uveitis.

91- The method of claim 86, wherein said amount is at least from about 10 and 80 mg/day.

92- The method of claim 86, wherein said amount is at least from about 20 and 40mg/day.

93- Use of a statin or a functionally or structurally equivalent molecule in the preparation of a medicament for reducing inflammation in an inflammatory ocular disorder, such statin being present in an amount effective for reducing inflammation.
